(19) United States
(12) Patent Application Publication
(10) Pub. No.: US 2025/0009765 A1
(43) Pub. Date: Jan. 9, 2025

(54) COMPOSITIONS AND METHODS FOR REDUCING SLEEP DISTURBANCES AND TREATING SLEEP DISORDERS

(71) Applicant: FloraScience Inc., Milwaukie, OR (US)

(72) Inventors: Robert Paul Jensen, Portland, OR (US); Vaughn Hartung, Milwaukie, OR (US); Alleh Lindquist, Milwaukie, OR (US)

(21) Appl. No.: 18/444,077

(22) Filed: Feb. 16, 2024

Related U.S. Application Data
(60) Provisional application No. 63/523,786, filed on Jun. 28, 2023.

(51) Int. Cl. A61K 31/00; A61K 9/48; A61P 25/20

(52) U.S. Cl. CPC A61K 31/658; A61K 9/4825; A61P 25/20

(57) ABSTRACT

Provided are compositions containing cannabinol, or a pharmaceutically acceptable salt thereof, and methods of reducing sleep disturbances in a subject, comprising administering the cannabinol composition to the subject. Also provided are methods of treating sleep disorders in a subject, comprising administering the cannabinol composition to the subject.

### PRIORITY

This application claims the benefit of priority to U.S. Patent Application Ser. No. 63/523,786, filed on Jun. 28, 2023, the entire contents of which are hereby incorporated by reference.

### TECHNICAL FIELD

This disclosure relates to compositions containing cannabinol, or a pharmaceutically acceptable salt thereof, and methods of reducing sleep disturbances in a subject, comprising administering the cannabinol composition to the subject. The disclosure also relates to methods of treating sleep disorders in a subject, comprising administering the cannabinol composition to the subject.

### BACKGROUND

Difficulty falling asleep or remaining asleep is a significant medical issue that arises for a variety of reasons. These problems can arise from endogenous conditions such as sleep apnea, insomnia, or other sleep disorders. Other times, these problems arise from exogenous stresses such as the disruptive effect of shift work schedules, and “jet lag.” Whether caused by an endogenous or exogenous source, difficulty falling asleep or remaining asleep can result in problem sleepiness, which impairs the health, quality of life and safety of those affected. Current treatment methods have numerous drawbacks, including rebound insomnia, delayed onset of desired sedative effects, persistence of sedative effects after the desired sleep period, and side effects due to nonspecific activity such as psychomotor and memory deficits, myorelaxation, and disturbed sleep architecture, including REM sleep inhibition. Additionally, sedatives and hypnotics can be habit forming, can lose their effectiveness after extended use and may be metabolized more slowly by some people. There are an increasing number of people who have trouble sleeping at night and this trend is expected to continue.

Melatonin supplements can be helpful in treating sleep disorders, such as delayed sleep phase and can provide some relief from insomnia and jet lag. However, melatonin is associated with dependence and a variety of side effects, such as headache, dizziness, nausea, and daytime drowsiness. Cannabidiol (CBD) can be used to counteract the “hangover” effect that people experience with melatonin. Currently, the effects of CBD in sleep lacks solid clinical research studies.

Cannabinol (CBN) is another cannabinoid derived from the Cannabis plant and has been shown to be a sedative without any of the psychoactive properties of THC. There is also a lack of research and understanding of how cannabinol (CBN) interacts with the body, and, particularly, its effects as a sleep aid (Corroon, J. “Cannabinol and Sleep: Separating Fact from Fiction,” Cannabis and Cannabinoid Research, Volume 6, Number 5, 2021). Studies specifically assessing subjective effects associated with sleep, such as sedation or fatigue, are rare. From a mechanistic perspective, a low-affinity partial agonist of the CB1 receptor, like CBN, could cause sedation, as evidenced by experimental models investigating the effects of THC, another low-affinity partial agonist. This effect has only been demonstrated in some, not all, pre-clinical studies, and in only one clinical study. As of recently, there was insufficient published evidence to support a health claim related to sleep.

### SUMMARY

Provided in the present disclosure are methods of reducing sleep disturbances in a subject, comprising administering a composition comprising about 25 mg to about 100 mg of cannabinol, or a pharmaceutically acceptable salt thereof, to the subject. Also provided in the present disclosure are methods of reducing sleep disturbances in a subject, comprising administering a composition comprising about 40 mg to about 60 mg of cannabinol, or a pharmaceutically acceptable salt thereof, to the subject. In some embodiments, the composition comprises about 0.5 wt % or less of cannabidiol, or a pharmaceutically acceptable salt thereof, and about 0.5 wt % or less of Δ⁹⁽¹⁰⁾-THC.

Also provided herein are methods for treating a sleep disorder in a subject, comprising administering a composition comprising about 25 mg to about 100 mg of cannabinol, or a pharmaceutically acceptable salt thereof, to the subject. Provided herein are methods for treating a sleep disorder in a subject, comprising administering a composition comprising about 40 mg to about 60 mg of cannabinol, or a pharmaceutically acceptable salt thereof, to the subject. In some embodiments, the composition comprises about 0.5 wt % or less of cannabidiol and about 0.5 wt % or less of Δ⁹⁽¹⁰⁾-THC.

Also provided herein is a dosage form comprising cannabinol, or a pharmaceutically acceptable salt thereof, in an amount of about 25 mg to about 100 mg, wherein the cannabinol, or pharmaceutically acceptable salt thereof has a purity of at least about 90%. Provided herein is a dosage form comprising cannabinol, or a pharmaceutically acceptable salt thereof, in an amount of about 40 mg to about 60 mg, wherein the cannabinol, or pharmaceutically acceptable salt thereof has a purity of at least about 90%.

Also provided herein is a dosage form comprising cannabinol, or a pharmaceutically acceptable salt thereof, in an amount of about 25 mg to about 100 mg, wherein the cannabinol, or pharmaceutically acceptable salt thereof, has a purity of at least about 95%. Also provided herein is a dosage form comprising cannabinol, or a pharmaceutically acceptable salt thereof, in an amount of about 40 mg to about 60 mg, wherein the cannabinol, or pharmaceutically acceptable salt thereof, has a purity of at least about 90%.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 depicts the clinical study timeline.

FIG. 2. shows a map of the continental United States with the geographical location of each study participant indicated as a dot.

FIG. 3 depicts the PROMIS™ Sleep Disturbance 8A survey questions.

FIG. 4 depicts the PROMIS™ Sleep Disturbance SF4A survey questions.

FIG. 5 is a line graph showing the PROMIS™ Sleep Disturbance 8A score over time by study arm.

FIG. 6 shows the risk ratios for achieving minimum clinically important difference (MCID) in PROMIS™ Sleep Disturbance 8A score by study arm.

FIG. 7 shows the side-effect count reported by study participants in the different study arms.

### DETAILED DESCRIPTION

Provided in the present disclosure is a composition containing cannabinol, or a pharmaceutically acceptable salt thereof. In some embodiments, the composition contains high purity cannabinol, or a pharmaceutically acceptable salt thereof. Also provided are methods of reducing sleep disturbances in a subject comprising administering a composition containing cannabinol, or a pharmaceutically acceptable salt thereof, to the subject. In some embodiments, the cannabinol, or a pharmaceutically acceptable salt thereof, is administered in a dosage form containing about 25 mg to about 100 mg of cannabinol, or a pharmaceutically acceptable salt thereof (e.g., the cannabinol composition). In some embodiments, the cannabinol, or a pharmaceutically acceptable salt thereof, is administered in a dosage form containing about 40 mg to about 60 mg of cannabinol, or a pharmaceutically acceptable salt thereof (e.g., the cannabinol composition). In some embodiments, the cannabinol, or a pharmaceutically acceptable salt thereof, is administered in a dosage form containing about 25 mg of cannabinol, or a pharmaceutically acceptable salt thereof (e.g., the cannabinol composition). In some embodiments, the cannabinol, or a pharmaceutically acceptable salt thereof, is administered in a dosage form containing about 50 mg of cannabinol, or a pharmaceutically acceptable salt thereof (e.g., the cannabinol composition). In some embodiments, the cannabinol, or a pharmaceutically acceptable salt thereof, is administered in a dosage form containing about 100 mg of cannabinol, or a pharmaceutically acceptable salt thereof (e.g., the cannabinol composition).

The cannabinol, or a pharmaceutically acceptable salt thereof, in the compositions of the present disclosure has high purity and is substantially free of other cannabinoids, such as THC and CBD. For example, the cannabinol, or a pharmaceutically acceptable salt thereof, has a purity of at least about 90%, at least about 95%, at least about 98%, or at least about 99%.

Advantageously, it was found herein when subjects were administered the cannabinol composition, particularly that were administered high purity cannabinol in an amount of about 40 mg to about 60 mg (e.g., cannabinol having a purity of at least about 90%, at least about 95%, at least about 98%, or at least about 99%), the subjects experienced improved quality sleep (e.g., less sleep disturbances according to a PROMIS™ Sleep Disturbance 8a Test) as compared to subjects that were not administered the cannabinol composition (a placebo), and subjects that were administered a lower dose of CBN or a higher dose of CBN.

Also provided herein are methods of treating sleep disorders in a subject comprising administering the cannabinol composition of the present disclosure to the subject. In some embodiments, the cannabinol composition includes high purity cannabinol, or a pharmaceutically acceptable salt thereof, in an amount of about 25 mg to about 100 mg. In some embodiments, the cannabinol composition comprises high purity cannabinol, or a pharmaceutically acceptable salt thereof, in an amount of about 40 mg to about 60 mg. In some embodiments, the cannabinol composition comprises high purity cannabinol, or a pharmaceutically acceptable salt thereof, in an amount of about 25 mg. In some embodiments, the cannabinol composition comprises high purity cannabinol, or a pharmaceutically acceptable salt thereof, in an amount of about 50 mg. In some embodiments, the cannabinol composition comprises high purity cannabinol, or a pharmaceutically acceptable salt thereof, in an amount of about 100 mg.

### Definitions

Unless otherwise defined, all technical and scientific terms used in this document have the same meaning as commonly understood by one of ordinary skill in the art to which the present application belongs. Methods and materials are described in this document for use in the present application; other, suitable methods and materials known in the art can also be used. The materials, methods, and examples are illustrative only and not intended to be limiting. All publications, patent applications, patents, sequences, database entries, and other references mentioned in this document are incorporated by reference in their entirety. In case of conflict, the present specification, including definitions, will control.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, and 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about,” as used in this disclosure, can allow for a degree of variability in a value or range, for example, within 5%, or within 1% of a stated value or of a stated limit of a range.

As used in this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading can occur within or outside of that particular section.

As used herein, the term “cannabinoid” refers to a compound that is structurally related to the terpophenolic compounds metabolically produced by Cannabis sativa. Cannabinoids include the endocannabinoids (produced naturally in the body by humans and animals), the phytocannabinoids (found in Cannabis and some other plants), and synthetic cannabinoids. For example, a notable phytocannabinoid is Δ⁹⁽¹⁰⁾-tetrahydrocannabinol (Δ⁹⁽¹⁰⁾-THC), the primary psychoactive compound of Cannabis. Cannabidiol is another major constituent of the plant, representing up to 40% in extracts of the plant resin. There are at least 85 different cannabinoids isolated from Cannabis, which exhibit varied effects.

As used herein, the term “substantially free of” an ingredient(s) as provided throughout the disclosure is intended to mean that the composition or compound(s) contain less than about 0.1 wt % (percent by weight of the total weight of the composition or compound(s)), or insignificant or negligible amounts of said ingredient(s) unless specifically indicated otherwise. In some embodiments, the composition or dosage form of the present disclosure are substantially free of THC, meaning that the compositions or dosage forms, contain less than about 0.1 wt % THC. In some embodiments, the CBN, or a pharmaceutically acceptable salt thereof, and is substantially free of THC. In some embodiments, the CBN, or a pharmaceutically acceptable salt thereof, of the present disclosure is substantially free of Δ⁹⁽¹⁰⁾-THC, meaning that the CBN, or a pharmaceutically acceptable salt thereof, contains less than about 0.1 wt % Δ⁹⁽¹⁰⁾-THC.

The term “pharmaceutically acceptable salt” refers to a formulation of a compound that does not cause significant irritation to an organism to which it is administered and does not abrogate the biological activity and properties of the compound.

The “PROMIS™ Sleep Disturbance 8a Test” is a sleep score test given to subject(s) of a study according to the following reference: Purvis, Taylor E. BA; Neuman, Brian J. MD; Riley, Lee H. III MD; Skolasky, Richard L. ScD, “Discriminant Ability, Concurrent Validity, and Responsiveness of PROMIS Health Domains Among Patients With Lumbar Degenerative Disease Undergoing Decompression With or Without Arthrodesis,” SPINE: Nov. 1, 2018—Volume 43—Issue 21—p 1512-1520.

As used herein, “administering to a patient” refers to the process of introducing a composition or dosage form into the patient via an art-recognized means of introduction.

As used herein the term “disorder” is intended to be generally synonymous, and is used interchangeably with, the terms “disease,” “syndrome,” and “condition” (as in medical condition), in that all reflect an abnormal condition of the human or animal body or of one of its parts that impairs normal functioning, is typically manifested by distinguishing signs and symptoms.

As used herein, a “dose” means the measured quantity of an active agent to be taken at one time by a patient. In certain embodiments, wherein the active agent is not cannabinol free base, the quantity is the molar equivalent to the corresponding amount of cannabinol free base. For example, often a drug is packaged in a pharmaceutically acceptable salt form and the dosage for strength refers to the mass of the molar equivalent of the corresponding free base, cannabinol.

As used herein, “dosing regimen” means the dose of an active agent taken at a first time by a patient and the interval (time or symptomatic) at which any subsequent doses of the active agent are taken by the patient such as from about 25 mg to about 100 mg, or about 40 mg to about 60 mg once daily, e.g., about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg, once daily.

As used herein, “patient” or “individual” or “subject” means a mammal, including a human, for whom or which therapy is desired, and generally refers to the recipient of the therapy.

As used herein, “pharmaceutically acceptable” refers to a material that is not biologically or otherwise undesirable, i.e., the material can be incorporated into a composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” (or “active”) derivative or analog, refers to a derivative or analog having the same type of pharmacological activity as the parent compound and approximately equivalent in degree. The term “pharmaceutically acceptable salts” includes acid addition salts which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

As used herein, “treating” or “treatment” refers to therapeutic applications to slow or stop progression of a disorder, prophylactic application to prevent development of a disorder, and/or reversal of a disorder. Reversal of a disorder differs from a therapeutic application which slows or stops a disorder in that with a method of reversing, not only is progression of a disorder completely stopped, cellular behavior is moved to some degree, toward a normal state that would be observed in the absence of the disorder.

### Methods

Provided in the present disclosure are methods of reducing sleep disturbances in a subject, comprising administering a composition comprising about 25 mg to about 100 mg cannabinol, or a pharmaceutically acceptable salt thereof (e.g., the cannabinol composition), to the subject, wherein the composition comprises about 0.5 wt % or less of cannabidiol and about 0.5 wt % or less of Δ⁹⁽¹⁰⁾-THC. In some embodiments, the method includes administering a composition comprising about 40 mg to about 60 mg cannabinol, or a pharmaceutically acceptable salt thereof (e.g., the cannabinol composition), to the subject, wherein the composition comprises about 0.5 wt % or less of cannabidiol and about 0.5 wt % or less of Δ⁹⁽¹⁰⁾-THC.

Also provided herein are methods for treating a sleep disorder in a subject, comprising administering a composition comprising about 25 mg to about 100 mg cannabinol, or a pharmaceutically acceptable salt thereof, to the subject, wherein the composition comprises about 0.5 wt % or less of cannabidiol and about 0.5 wt % or less of Δ⁹⁽¹⁰⁾-THC. In some embodiments, the method includes administering a composition comprising about 40 mg to about 60 mg cannabinol, or a pharmaceutically acceptable salt thereof, to the subject, wherein the composition comprises about 0.5 wt % or less of cannabidiol and about 0.5 wt % or less of Δ⁹⁽¹⁰⁾-THC.

In some embodiments, the subject exhibits one or more risk factors or symptoms associated with lack of sleep or a sleep disorder.

In some embodiments, the sleep disorder is selected from the group of: insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition.

In some embodiments, the subject experiences less sleep disturbances according to a PROMIS™ Sleep Disturbance 8a Test after administering the cannabinol composition as compared to before administering the cannabinol composition.

Cannabinol (CBN), chemical name 6,6,9-trimethyl-3-pentyl-benzo[c]chromen-1-ol, has a chemical structure as follows:

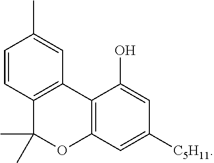

The cannabinol compositions of the disclosure can include about 25 mg to about 100 mg of cannabinol, or a pharmaceutically acceptable salt thereof, such as about 25 mg to about 90 mg, about 25 mg to about 80 mg, about 25 mg to about 75 mg, about 25 mg to about 70 mg, about 25 mg to about 60 mg, about 25 mg to about 50 mg, about 25 mg to about 40 mg, about 25 mg to about 30 mg, about 30 mg to about 100 mg, about 30 mg to about 90 mg, about 30 mg to about 80 mg, about 30 mg to about 75 mg, about 30 mg to about 70 mg, about 30 mg to about 60 mg, about 30 mg to about 50 mg, about 30 mg to about 40 mg, about 40 mg to about 100 mg, about 40 mg to about 90 mg, about 40 mg to about 80 mg, about 40 mg to about 75 mg, about 40 mg to about 70 mg, about 40 mg to about 60 mg, about 40 mg to about 50 mg, about 50 mg to about 100 mg, about 50 mg to about 90 mg, about 50 mg to about 80 mg, about 50 mg to about 75 mg, about 50 mg to about 70 mg, about 50 mg to about 60 mg, about 60 mg to about 100 mg, about 60 mg to about 90 mg, about 60 mg to about 80 mg, about 60 mg to about 75 mg, about 60 mg to about 70 mg, about 70 mg to about 100 mg, about 70 mg to about 90 mg, about 70 mg to about 80 mg, about 70 mg to about 75 mg, about 75 mg to about 100 mg, about 75 mg to about 90 mg, about 75 mg to about 80 mg, about 80 mg to about 100 mg, about 80 mg to about 90 mg, or about 90 mg to about 100 mg. For example, the cannabinol composition can include about 25, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of cannabinol, or a pharmaceutically acceptable salt thereof. In some embodiments, the cannabinol composition includes about 25 mg to about 100 mg of cannabinol, or a pharmaceutically acceptable salt thereof. In some embodiments, the cannabinol composition includes about 40 mg to about 60 mg of cannabinol, or a pharmaceutically acceptable salt thereof. In some embodiments, the cannabinol composition includes about 25 mg of cannabinol, or a pharmaceutically acceptable salt thereof. In some embodiments, the cannabinol composition includes about 50 mg of cannabinol, or a pharmaceutically acceptable salt thereof. In some embodiments, the cannabinol composition includes about 100 mg of cannabinol, or a pharmaceutically acceptable salt thereof.

In some embodiments, the cannabinol, or a pharmaceutically acceptable salt thereof, of the cannabinol composition of the present disclosure has high purity (e.g., a purity of at least about 90%, at least about 95%, at least about 98%, or at least about 99%). For example, the cannabinol, or a pharmaceutically acceptable salt thereof, of the cannabinol composition can be at least 95%, 95.5%, 96%, 96.5%, 97%, 97.5%, 98%, 98.5%, 99%, 99.5%, 99.6%, 99.7%, 99.8%, 99.9%, or 99.99% pure. In some embodiments, the cannabinol, or a pharmaceutically acceptable salt thereof, of the cannabinol composition has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the cannabinol, or a pharmaceutically acceptable salt thereof, of the cannabinol composition has a purity of at least about 99%. In some embodiments, the cannabinol, or a pharmaceutically acceptable salt thereof, of the cannabinol composition has a purity of at least about 99.5%. In some embodiments, the cannabinol, or a pharmaceutically acceptable salt thereof, of the cannabinol composition has a purity of at least about 99.9%.

In some embodiments, the cannabinol composition contains about 0.5 wt % THC or less, such as about 0.45 wt %, about 0.4 wt %, about 0.35 wt %, about 0.3 wt %, about 0.25 wt %, about 0.2 wt %, about 0.15 wt %, about 0.1 wt % THC, or less. In some embodiments, the cannabinol composition contains less than the legal limit of THC, such as less than 0.3 wt % THC. In some embodiments, the cannabinol composition is substantially free of THC. In some embodiments, the THC is Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ^{10(10a)}-tetrahydrocannabinol (Δ^{10(10a)}-THC), or Δ^{6a(10a)}-tetrahydrocannabinol (Δ^{6a(10a)}-THC), or combinations thereof. In some embodiments, the cannabinol composition contains about 0.5 wt % or less of Δ⁹⁽¹⁰⁾-THC. In some embodiments, the cannabinol composition, contains less than the legal limit of Δ⁹⁽¹⁰⁾-THC, such as less than 0.3 wt % Δ⁹⁽¹⁰⁾-THC. In some embodiments, the cannabinol composition is substantially free of Δ⁹⁽¹⁰⁾-THC. Advantageously, the cannabinol composition of the methods of the present disclosure can contain THC (e.g., Δ⁹⁽¹⁰⁾-THC) in such an insignificant amount that the psychoactive effects of certain forms of THC (e.g., Δ⁹⁽¹⁰⁾-THC) are diminished or are completely absent when a subject is administered the cannabinol composition.

In some embodiments, the cannabinol composition contains about 0.5 wt % cannabidiol or less, such as about 0.45 wt %, about 0.4 wt %, about 0.35 wt %, about 0.3 wt %, about 0.25 wt %, about 0.2 wt %, about 0.15 wt %, about 0.1 wt % cannabidiol, or less. In some embodiments, the cannabinol composition contains less than 0.3 wt % cannabidiol. In some embodiments, the cannabinol composition is substantially free of cannabidiol. Advantageously, the cannabinol composition, of the methods of the present disclosure contains cannabidiol in such an insignificant amount that the psychoactive effects of certain forms of cannabidiol are diminished or are completely absent when a subject is administered the cannabinol composition.

In some embodiments, the cannabinol composition contains about 0.5 wt % or less of additional cannabinoids (e.g., the cannabinoids listed in Table 1) other than CBN, such as about 0.45 wt %, about 0.4 wt %, about 0.35 wt %, about 0.3 wt %, about 0.25 wt %, about 0.2 wt %, about 0.15 wt %, about 0.1 wt %, or less of additional cannabinoids. In some embodiments, the cannabinol composition contains less than 0.3 wt % of additional cannabinoids. In some embodiments, the cannabinol composition is substantially free of additional cannabinoids.

Examples of additional cannabinoids are described in Table 1.

TABLE 1
Additional Cannabinoids

| Trivial Name | Structure | IUPAC name |
|---|---|---|
| Cannabichromene (CBC) | 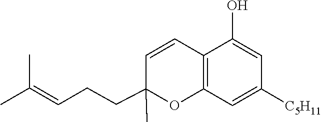 | 2-Methyl-2-(4-methylpent- 3-enyl)-7-penyl-5- chromenol |
| Cannabichromenic acid (CBCA) | 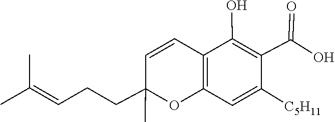 | 5-Hydroxy-2-methyl-2-(4- methylpent-3-enyl)-7- penylchromene-6- carboxylic acid |
| Cannabichromenevarin (CBCV) | 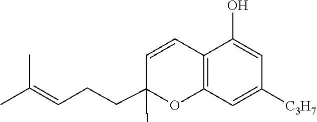 | 2-Methyl-2-(4-methylpent- 3-enyl)-7-propyl-5- chromenol |
| Cannabidiol (CBD) | 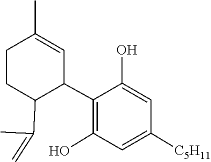 | 2-[(1R,6R)-6-Isopropenyl- 3-methylcyclohex-2-en-1- yl]-5-pentylbenzene-1,3- diol |
| 11-Hydroxycannabidiol (11-OH-CBD) | 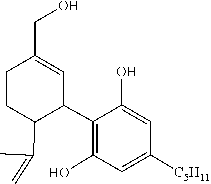 | 5′-(hydroxymethyl)-4- pentyl-2′-(prop-1-en-2-yl)- 1′,2′,3′,4′-tetrahydro-[1,1′- biphenyl]-2,6-diol |
| Cannabidiolic acid (CBDA) | 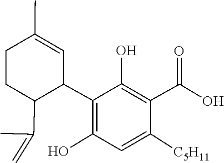 | (1′R,2′R)-2,6-Dihydroxy- 5′-methyl- 4-pentyl-2′-(prop-1-en-2- yl)-1′,2′,3′,4′- tetrahydro[1,1′-biphenyl]- 3-carboxylic acid |
| Cannabidivarol (CBDV) | 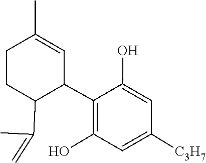 | 2-[(1R,6R)-6-Isopropenyl- 3-methylcyclohex-2-en-1- yl]-5-propylbenzene-1,3- diol |
| Cannabielsoin (CBE) | 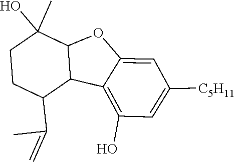 | (5aS,6S,9R,9aR)-6-methyl- 3- pentyl-9-prop-1-en-2-yl- 7,8,9,9a-tetrahydro-5aH- dibenzofuran- 1,6-diol |
| Cannabifuran (CBF) | 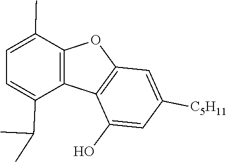 | 5-isopropyl-8-methyl-2- pentyl-9- oxa-4-fluorenol |
| Cannabigerol (CBG) | 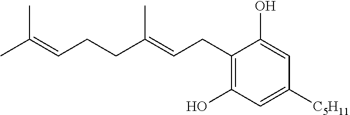 | 2-[(2E)-3,7-Dimethylocta- 2,6- dienyl]-5-penyl-benzene- 1,3- diol |
| Cannabigerolic acid (CBGA) | 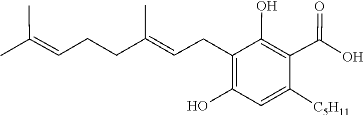 | 3-[(2E)-3,7-Dimethylocta- 2,6- dien-1-yl]-2,4-dihydroxy- 6-pentylbenzoic acid |
| Cannabigerovarinol (CBGV) | 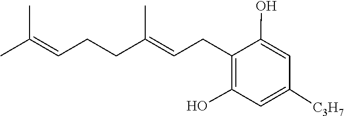 | 2-[(2E)-3,7-Dimethylocta- 2,6-dienyl]-5-propyl- benzene-1,3- diol |
| Cannabicyclol (CBL) | 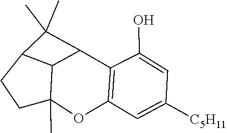 | (1aR-(1a alpha,3a alpha,8b alpha,8calpha))- 1a,2,3,3a,8b,8c- hexahydro-1,1,3a-trimethyl- 6-pentyl-1H-4- oxabenzo(f)- cyclobut(cd)inden-8-ol |
| 11-Hydroyxcannabinol (11-OH-CBN) | 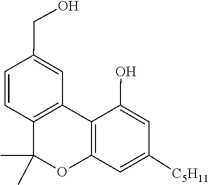 | 9-(hydroxymethyl)-6,6- dimethyl-3-pentyl-6H- benzo[c]chromen-1-ol |
| Cannabinolic acid (CBNA) | 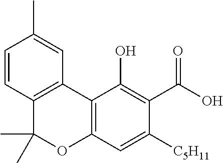 | 1-hydroxy-6,6,9-trimethyl- 3- pentyl-benzo[c]chromene- 2-carboxylic acid |
| Cannabivarol (CBNV) | 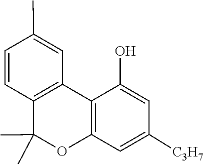 | 6,6,9-trimethyl-3-pentyl- benzo[c]chromen-1-ol |
| Acetylcannabinol (CBN- OAc) | 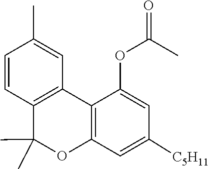 | 1-acetoxy-6,6,9-trimethyl- 3- pentyl-benzo[c]chromene |
| Methoxycannabinol (CBN-OMe) | 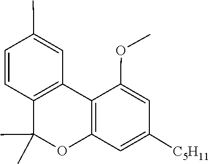 | 1-methoxy-6,6,9-trimethyl- 3- pentyl-benzo[c]chromene |
| CBN-OTMS | 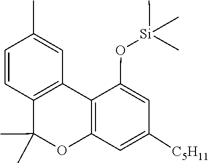 | trimethyl((6,6,9-trimethyl- 3-pentyl-6H- benzo[c]chromen-1- yl)oxy)silane |
| Cannabinodiol (CBND) | 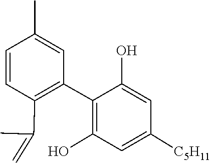 | 2,6-dihydroxy-4-pentyl-2′- isopropenyl-5′-methyl- 1,1′-biphenyl |
| Cannabicitran (CBT) | 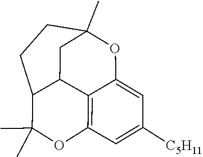 | 1,5,5-trimethyl-9- pentyl-6,15- dioxatetracyclo [9.3.1.0^{4,13}.0^{7,12}]- pentadeca- 7(12),8,10-triene |
| Dehydrocannabifuran (DHCBF) | 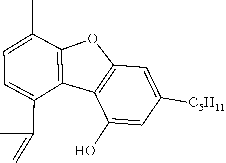 | 5-isopropenyl-8-methyl-2- pentyl-9-oxa-4-fluorenol |
| Dihydrocannabinodiol (H₂CBND) | 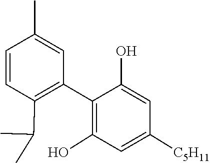 | 2,6-dihydroxy-4-pentyl-2′- isopropyl-5′-methyl-1,1′- biphenyl |
| Dihydrocannabidiol (H₂CBD) | 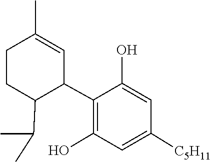 | 2-[(1R,6R)-6-Isopropyl-3- methylcyclohex-2-en-1- yl]-5-pentylbenzene-1,3- diol |
| Tetrahydrocannabidiol (H₄CBD) | 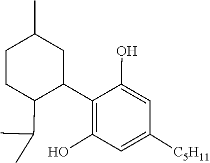 | 2-[(1S,6R)-6-Isopropyl-3- methylcyclohexyl]-5- pentylbenzene-1,3-diol |
| Δ⁸⁽⁹⁾-iso- tetrahydrocannabinol (Δ⁸⁽⁹⁾-iso-THC) | 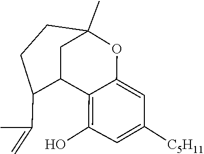 | 3,4,5,6-tetrahydro-2- methyl-5-(1- methylethenyl)-9-pentyl- 2,6-methano-2H-1- benzoxocin-7-ol |
| Δ⁴⁽⁸⁾-iso- tetrahydrocannabinol (Δ⁴⁽⁸⁾-iso-THC) | 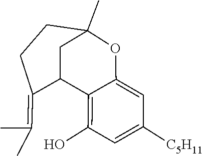 | 3,4,5,6-tetrahydro-2- methyl-5-(1- methylethylidene)-9- pentyl-2,6-methano-2H-1- benzoxocin-7-ol |
| Δ⁴⁽⁵⁾-iso- tetrahydrocannabinol (Δ⁴⁽⁵⁾-iso-THC) | 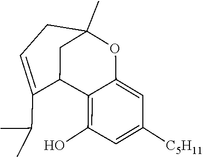 | 3,6-dihydro-2-methyl-5-(1- methylethyl)-9-pentyl-2,6- methano-2H-1- benzoxocin-7-ol |
| Hexahydrocannabinol (HHC) | 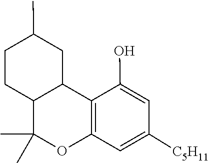 | 6,6,9-trimethyl-3-pentyl- 6a,7,8,9,10,10a-hexahydro- 6H-benzo[c]chromen-1-ol |
| Δ⁸⁽⁹⁾- tetrahydrocannabinol (Δ⁸⁽⁹⁾-THC) | 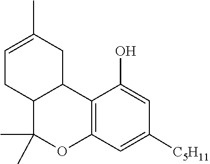 | 6,6,9-trimethyl-3-pentyl- 6a,7,10,10a-tetrahydro- 6H-benzo[c]chromen-1-ol |
| 11-hydroxy-Δ⁸⁽⁹⁾- tetrahydrocannabinol (11-OH-Δ⁸⁽⁹⁾-THC) | 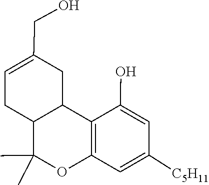 | 9-(hydroxymethyl)-6,6- dimethyl-3-pentyl- 6a,7,10,10a-tetrahydro-6H- benzo[c]chromen-1-ol |
| Δ⁸⁽⁹⁾- tetrahydrocannabinolic acid (Δ⁸⁽⁹⁾-THCA) | 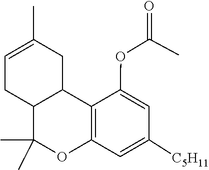 | 6,6,9-trimethyl-3-pentyl- 6a,7,10,10a-tetrahydro-6H- benzo[c]chromen-1-yl acetate |
| Methoxy Δ⁸⁽⁹⁾- tetrahydrocannabinol (Δ⁸⁽⁹⁾-THC-OMe) | 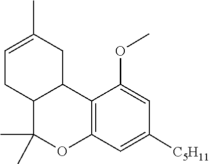 | 1-methoxy-6,6,9-trimethyl- 3-pentyl-6a,7,10,10a- tetrahydro-6H- benzo[c]chromene |
| Δ⁸⁽⁹⁾- tetrahydrocannabivarol (Δ⁸⁽⁹⁾-THCV) | 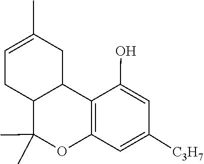 | 6,6,9-trimethyl-3-propyl- 6a,7,10,10a-tetrahydro-6H- benzo[c]chromen-1-ol |
| Δ⁹⁽¹⁰⁾- tetrahydrocannabinol (Δ⁹⁽¹⁰⁾-THC) | 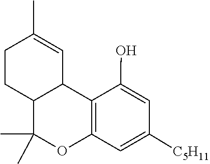 | 6,6,9-trimethyl-3-pentyl- 6a,7,8,10a-tetrahydro- 6H-benzo[c]chromen-1- ol |
| 11-hydroxy-Δ⁹⁽¹⁰⁾- tetrahydrocannabinol (11-OH-Δ⁹⁽¹⁰⁾-THC) | 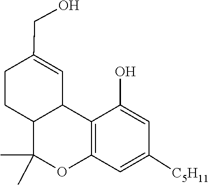 | 9-(hydroxymethyl)-6,6,- dimethyl-3-pentyl- 6a,7,8,10a-tetrahydro- 6H-benzo[c]chromen-1- ol |
| Δ⁹⁽¹⁰⁾- tetrahydrocannabinolic acid (Δ⁹⁽¹⁰⁾-THCA) | 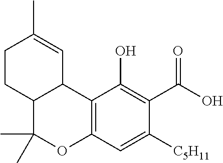 | (6aR,10aR)-1-Hydroxy- 6,6,9-trimethyl-3-pentyl- 6a,7,8,10a-tetrahydro-6H- benzo[c]chromene-2- carboxylic acid |
| Δ⁹⁽¹⁰⁾- tetrahydrocannabinovarol (Δ⁹⁽¹⁰⁾-THCV) | 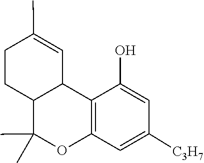 | 6,6,9-Trimethyl-3-propyl- 6a,7,8,10a-tetrahydro-6H- benzo[c]chromen-1-ol |
| Δ^{10(10a)}- tetrahydrocannabinol (Δ^{10(10a)}-THC) | 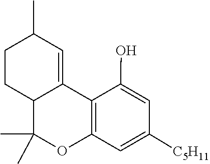 | 6,6,9-trimethyl-3-pentyl- 6a,7,8,9-tetrahydro-6H- benzo[c]chromen-1-ol |
| Δ^{6a(10a)}- tetrahydrocannabinol (Δ^{6a(10a)}-THC) | 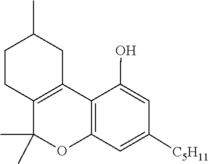 | 6,6,9-trimethyl-3-pentyl- 7,8,9,10-tetrahydro-6H- benzo[c]chromen-1-ol |
| Δ^{6a(10a)}-THC-OTMS | 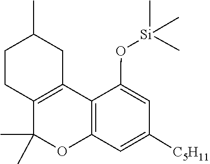 | trimethyl((6,6,9-trimethyl- 3-pentyl-7,8,9,10- tetrahydro-6H- benzo[c]chromen-1- yl)oxy)silane |
| Tetrahydrocannabigero (H₄CBG) | 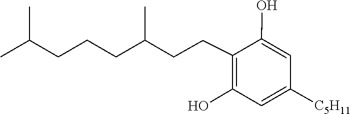 | 2-(3,7-dimethyloctyl)- 5- pentylbenzene-1,3-diol |

In some embodiments, the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H2CBND), dihydrocannabidiol (H2CBD), tetrahydrocannabidiol (H4CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), tetrahydrocannabigerol (H4CBG), and combinations thereof.

In some embodiments, the subject exhibits one or more risk factors or symptoms associated with lack of sleep or a sleep disorder.

In some embodiments, the sleep disorder is selected from the group consisting of insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition.

Also provided is a composition for reducing sleep disturbances in a subject, wherein the composition includes cannabinol, or a pharmaceutically acceptable salt thereof, in an amount of about 25 mg to about 100 mg, wherein the cannabinol, or pharmaceutically acceptable salt thereof, has a purity of at least about 95%. In some embodiments, the composition for reducing sleep disturbances in a subject includes cannabinol, or a pharmaceutically acceptable salt thereof, in an amount of about 40 mg to about 60 mg, wherein the cannabinol, or pharmaceutically acceptable salt thereof, has a purity of at least about 95%.

Also provided is a composition for treating sleep disorders in a subject, wherein the composition includes cannabinol, or a pharmaceutically acceptable salt thereof, in an amount of about 40 mg to about 60 mg, wherein the cannabinol, or pharmaceutically acceptable salt thereof, has a purity of at least about 95%. In some embodiments, the composition for treating sleep disorders in a subject includes cannabinol, or a pharmaceutically acceptable salt thereof, in an amount of about 40 mg to about 60 mg, wherein the cannabinol, or pharmaceutically acceptable salt thereof, has a purity of at least about 95%.

The compositions of the disclosure can include one or more pharmaceutical excipients. In some embodiments, the compositions of the disclosure include only cannabinol, or a pharmaceutically acceptable salt thereof.

The term “pharmaceutically acceptable excipient” refers to an excipient for administration of a pharmaceutical agent, such as a cannabinoid (e.g., cannabinol, or a pharmaceutically acceptable salt thereof) described herein. The term refers to any pharmaceutical excipient that can be administered without undue toxicity. Excipients include, but are not limited to, carriers, solvents, stabilizers, adjuvants, and diluents.

Suitable excipients can be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

The choice of excipient, to a large extent, depends on factors, such as the particular mode of administration, the effect of the excipient on the solubility and stability of the active ingredient, and the nature of the dosage form.

The pharmaceutical compositions provided herein can be provided in unit dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit dosage forms can be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The pharmaceutical compositions provided herein can be administered alone, or in combination with one or more other compounds provided herein, one or more other active ingredients. The pharmaceutical compositions provided herein can be formulated in various dosage forms for oral, parenteral, and topical administration. The pharmaceutical compositions can also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art). The pharmaceutical compositions provided herein can be administered at once, or multiple times at intervals of time. It is understood that the precise dosage and duration of treatment can vary with the age, weight, and condition of the patient being treated, and can be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### Oral Administration

The pharmaceutical compositions provided herein can be provided in solid, semisolid, or liquid dosage forms for oral administration. As used herein, oral administration also includes buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, PA); and mixtures thereof. Suitable fillers include, but are not limited to, talc, calcium carbonate, microcrystalline cellulose, powdered cellulose, dextrates, kaolin, mannitol, silicic acid, sorbitol, starch, pregelatinized starch, and mixtures thereof. The binder or filler can be present from about 50% to about 99% by weight in the compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Vee gum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the compositions provided herein varies upon the type of formulation, and is readily discernible to those of ordinary skill in the art. The compositions provided herein can contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL®200 (W.R. Grace Co., Baltimore, MD) and CAB-O-SIL® (Cabot Co. of Boston, MA); and mixtures thereof. The compositions provided herein can contain about 0.1% to about 5% by weight of a lubricant. Suitable glidants include colloidal silicon dioxide, CAB-O-SIL® (Cabot Co. of Boston, MA), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients can serve several functions, even within the same formulation. The compositions provided herein can be provided as compressed tablets, tablet triturates, chewable lozenges, rapidly dissolving tablets, multiple compressed tablets, or enteric-coating tablets, sugar-coated, or film-coated tablets. Enteric coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The compositions provided herein can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells can contain a preservative to prevent the growth of microorganisms. Suitable preservatives are those as described herein, including methyl- and propyl-parabens, and sorbic acid. The liquid, semisolid, and solid dosage forms provided herein can be encapsulated in a capsule. Suitable liquid and semisolid dosage forms include solutions and suspensions in propylene carbonate, vegetable oils, or triglycerides. The capsules can also be coated as known by those of skill in the art in order to modify or sustain dissolution of the active ingredient.

The compositions provided herein can be provided as food product, oil, or drink, in the form of gummies, creams, salves, tinctures, tablets, capsules, pastes, gelatin suppositories, crystals, ointments, jellies, e-juices, powders, pills, syrups, drops, patches, chewing gums, dried whole or partial plant parts, and dusts, such that it is safe for mammals and can provide assistance in dealing with various ailments and diseases.

The compositions provided herein can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and can also contain a preservative. For a liquid dosage form, for example, a solution in a polyethylene glycol can be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or polyalkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The compositions provided herein for oral administration can be also provided in the forms of liposomes, micelles, microspheres, or nanosystems.

The compositions provided herein can be provided as noneffervescent or effervescent, granules and powders, to be reconstituted into a liquid dosage form. Pharmaceutically acceptable carriers and excipients used in the non-effervescent granules or powders can include diluents, sweeteners, and wetting agents. Pharmaceutically acceptable carriers and excipients used in the effervescent granules or powders can include organic acids and a source of carbon dioxide. Coloring and flavoring agents can be used in all of the above dosage forms. The compositions provided herein can be formulated as immediate or modified release dosage forms, including delayed-, sustained, pulsed-, controlled, targeted-, and programmed-release forms.

The compositions provided herein can be co-formulated with other active ingredients which do not impair the desired therapeutic action, or with substances that supplement the desired action, such as antacids, proton pump inhibitors, and H2-receptor antagonists.

The compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

### Topical Administration

The compositions provided herein can be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The compositions can also be administered topically by electroporation, iontophoresis, phonophoresis, sonophoresis and microneedle or needle-free injection, such as POWDERJECT™ (Chiron Corp., Emeryville, CA), and BIOJECT™ (Bioject Medical Technologies Inc., Tualatin, OR).

The compositions provided herein can be provided in the forms of ointments, creams, and gels. Suitable ointment vehicles include oleaginous or hydrocarbon bases, including such as lard, benzoinated lard, olive oil, cottonseed oil, and other oils, white petrolatum; emulsifiable or absorption bases, such as hydrophilic petrolatum, hydroxystearin sulfate, and anhydrous lanolin; water-removable bases, such as hydrophilic ointment; water-soluble ointment bases, including polyethylene glycols of varying molecular weight; emulsion bases, either water-in-oil (W/O) emulsions or oil-in-water (O/W) emulsions, including cetyl alcohol, glyceryl monostearate, lanolin, and stearic acid. These vehicles are emollient but generally require addition of antioxidants and preservatives.

Suitable cream base can be oil-in-water or water-in-oil. Cream vehicles can be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation can be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, Carbopol®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include vehicles, such as stiffening agents, which produce a melting point in the proximity of body temperature, when formulated with the compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles can be used. Rectal and vaginal suppositories can be prepared by the compressed method or molding. The typical weight of a rectal and vaginal suppository is about 2 g to 3 g.

The compositions provided herein can be administered ophthalmically in the forms of solutions, suspensions, ointments, emulsions, gel-forming solutions, powders for solutions, gels, ocular inserts, and implants.

The compositions provided herein can be administered intranasally or by inhalation to the respiratory tract. The compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The compositions provided herein can be micronized to a size suitable for delivery by inhalation, such as 50 micrometers or less, or 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator can be formulated to contain a powder mix of the compositions provided herein; a suitable powder base, such as lactose or starch; and a performance modifier, such as /-leucine, mannitol, or magnesium stearate. The lactose can be anhydrous or in the form of the monohydrate. Other suitable excipients include dextran, glucose, maltose, sorbitol, xylitol, fructose, sucrose, and trehalose. The compositions provided herein for inhaled/intranasal administration can further comprise a suitable flavor, such as menthol and levomenthol, or sweeteners, such as saccharin or saccharin sodium.

The compositions provided herein for topical administration can be formulated to be immediate release or modified release, including delayed-, sustained-, pulsed-, controlled-, targeted, and programmed release.

### Modified Release

The compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms.

The compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

The compositions provided herein in a modified release dosage form can be fabricated using a matrix controlled release device known to those skilled in the art.

In certain embodiments, the compositions provided herein in a modified release dosage form is formulated using an erodible matrix device, which is water swellable, erodible, or soluble polymers, including synthetic polymers, naturally occurring polymers and derivatives, such as polysaccharides and proteins, and poly(cannabinoid)s, such as poly(CBD-adipate), poly(CBG-adipate), or polyesters of cannabinoids.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, NJ); poly(2-hydroxyethyl-methacrylate); polylactides; copolymers of L-glutamic acid and ethyl-L-glutamate; degradable lactic acid glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In certain embodiments, the compositions are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients in the compositions.

The compositions provided herein in a modified release dosage form can be prepared by methods known to those skilled in the art, including direct compression, dry or wet granulation followed by compression, melt-granulation followed by compression.

The compositions provided herein in a modified release dosage form can be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as Mannogeme EZ (SPI Pharma, Lewes, DE) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, Ca ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water, as disclosed in U.S. Pat. No. 5,798,119. Such hydrophobic but water-permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes. The delivery port(s) on the semipermeable membrane can be formed postcoating by mechanical or laser drilling. Delivery port(s) can also be formed in situ by erosion of a plug of water-soluble material or by rupture of a thinner portion of the membrane over an indentation in the core. In addition, delivery ports can be formed during coating process.

The total amount of the active ingredient(s) released and the release rate can substantially by modulated via the thickness and porosity of the semipermeable membrane, the composition of the core, and the number, size, and position of the delivery ports.

The compositions in an osmotic controlled-release dosage form can further comprise additional conventional excipients as described herein to promote performance or processing of the formulation.

The osmotic controlled-release dosage forms can be prepared according to conventional methods and techniques known to those skilled in the art.

In certain embodiments, the compositions provided herein are formulated as AMT controlled-release dosage form, which comprises an asymmetric osmotic membrane that coats a core comprising the active ingredient(s) and other pharmaceutically acceptable excipients. The AMT controlled-release dosage forms can be prepared according to conventional methods and techniques known to those skilled in the art, including direct compression, dry granulation, wet granulation, and a dip-coating method.

In certain embodiments, the compositions provided herein are formulated as ESC controlled-release dosage form, which comprises an osmotic membrane that coats a core comprising the active ingredient(s), hydroxylethyl cellulose, and other pharmaceutically acceptable excipients.

The compositions provided herein in a modified release dosage form can be fabricated a multiparticulate controlled release device, which comprises a multiplicity of particles, granules, or pellets, ranging from about 10 pm to about 3 mm, about 50 pm to about 2.5 mm, or from about 100 pm to 1 mm in diameter. Such multiparticulates can be made by the processes know to those skilled in the art, including wet- and dry-granulation, extrusion/spheronization, roller-compaction, melt-congealing, and by spray-coating seed cores.

Other excipients as described herein can be blended with the compositions to aid in processing and forming the multiparticulates. The resulting particles can themselves constitute the multiparticulate device or can be coated by various film forming materials, such as enteric polymers, water-swellable, and water-soluble polymers. The multiparticulates can be further processed as a capsule or a tablet.

In certain embodiments, the composition is administered orally.

In certain embodiments, the composition is administered in the form of a tablet or capsule. In some embodiments, the composition is administered in the form of a gel capsule. In some embodiments, the composition is administered in the form of a soft gel capsule.

In certain embodiments, the composition is administered with or without food.

### Dosage Form

Also provided herein is a dosage form comprising cannabinol, or a pharmaceutically acceptable salt thereof, in an amount of about 25 mg to about 100 mg, wherein the cannabinol, or pharmaceutically acceptable salt thereof, has a purity of at least about 95%. In some embodiments, the dosage form comprises cannabinol, or a pharmaceutically acceptable salt thereof, in an amount of about 40 mg to about 60 mg, wherein the cannabinol, or pharmaceutically acceptable salt thereof, has a purity of at least about 95%. In some embodiments, the dosage form comprises cannabinol, or a pharmaceutically acceptable salt thereof, in an amount of about 25 mg, wherein the cannabinol, or pharmaceutically acceptable salt thereof, has a purity of at least about 95%. In some embodiments, the dosage form comprises cannabinol, or a pharmaceutically acceptable salt thereof, in an amount of about 50 mg, wherein the cannabinol, or, or a pharmaceutically acceptable salt thereof pharmaceutically acceptable salt thereof, has a purity of at least about 95%. In some embodiments, the dosage form comprises cannabinol, or a pharmaceutically acceptable salt thereof, in an amount of about 40 mg to about 100 mg, wherein the cannabinol, or pharmaceutically acceptable salt thereof, has a purity of at least about 95%.

The cannabinol, or a pharmaceutically acceptable salt thereof, included in the dosage form of the present disclosure can be in an amount of about 25 mg to about 100 mg. In some embodiments, the cannabinol, or a pharmaceutically acceptable salt thereof, included in the dosage form of the present disclosure can be in an amount of about 40 mg to about 60 mg. For example, the cannabinol, or a pharmaceutically acceptable salt thereof, included in the dosage form can be in an amount of about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg. In some embodiments, the cannabinol, or a pharmaceutically acceptable salt thereof, included in the dosage form is in an amount of about 25 mg. In some embodiments, the cannabinol, or a pharmaceutically acceptable salt thereof, included in the dosage form is in an amount of about 50 mg. In some embodiments, the cannabinol, or a pharmaceutically acceptable salt thereof, included in the dosage form is in an amount of about 100 mg.

The dosage form of the present disclosure can be administered on a regimen of 1 to 4 times per day, including once, twice, three times, and four times per day. In some embodiments, dosage form of the present disclosure can be administered on a regimen of 1 time per day.

The dosage form of the present disclosure can be administered prior to the subject going to bed (e.g., when the subject first attempts to go to sleep for that day). In some embodiments, the dosage form is administered about 0.5 hours to about 5 hours prior to the subject going to bed. In some embodiments, the dosage form is administered about 1 hour to about 2 hours prior to the subject going to bed.

The dosage form of the disclosure can include the composition as disclosed herein.

In certain embodiments, the dosage level is about from 25 mg to 100 mg per day. In some embodiments, the dosage level is about from 40 mg to 60 mg per day. In some embodiments, the dosage level is about from 45 mg to 55 mg per day. In certain embodiments, the dosage level is about 25 mg per day. In certain embodiments, the dosage level is about 50 mg per day. In certain embodiments, the dosage level is about 100 mg per day.

For oral administration, the compositions can be provided in the form of capsules (e.g., gel capsules) containing about 1.0 mg to about 100 mg of the active ingredient, particularly about 1, about 5, about 10, about 15, about 20, about 25, about 30, about 40, about 45, about 50, about 55, about 60 mg, about 65 mg, about 70 mg, about 75 mg, about 80 mg, about 85 mg, about 90 mg, about 95 mg, or about 100 mg of the active ingredient for the symptomatic adjustment of the dosage to the patient to be treated. In certain embodiments, the compositions can be provided in the form of capsules (e.g., gel capsules) containing about 25 mg of the active ingredient. In certain embodiments, the compositions can be provided in the form of capsules (e.g., gel capsules) containing about 50 mg of the active ingredient. In certain embodiments, the compositions can be provided in the form of capsules (e.g., gel capsules) containing about 100 mg of the active ingredient. In certain embodiments, the compositions can be provided in the form of capsules (e.g., gel capsules) containing about 25 mg of the active ingredient and two can be administered in one dose. In certain embodiments, the compositions can be provided in the form of capsules (e.g., gel capsules) containing about 25 mg of the active ingredient and four can be administered in one dose.

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Combination Therapies

The cannabinol, or a pharmaceutically acceptable salt thereof, of the disclosure can also be combined or used in combination with other agents useful in reducing sleep disturbances or the treatment, prevention, or amelioration of one or more symptoms of the diseases or conditions for which the cannabinol, or a pharmaceutically acceptable salt thereof, provided herein is useful, including sleep disorders and other conditions commonly treated with sleep medication.

In certain embodiments, the cannabinol, or a pharmaceutically acceptable salt thereof, provided herein can also be combined or used in combination with another sleep drug. In certain embodiments, the sleep drug is melatonin, diphenhydramine, doxylamine, valerian, or prescription sleep medication, such as, daridorexant, doxepin, eszopiclone, lemborexant, ramelteon, suvorexant, temazepam, triazolam, zaleplon, and zolpidem. In certain embodiments, the cannabinol, or a pharmaceutically acceptable salt thereof, of the disclosure is used in combination with melatonin. In some embodiments, the cannabinol, or a pharmaceutically acceptable salt thereof, of the disclosure is used in combination with additional cannabinoids, such as one or more cannabinoids listed in Table 1.

Such other agents, or drugs, can be administered, by a route and in an amount commonly used thereof, simultaneously or sequentially with the compounds provided herein. When compounds provided herein are used contemporaneously with one or more other drugs, a composition containing such other drugs in addition to the compounds provided herein can be utilized, but is not required. Accordingly, the compositions provided herein include those that also contain one or more other active ingredients or therapeutic agents, in addition to the compounds provided herein.

The weight ratio of the compounds provided herein to the second active ingredient can be varied, and will depend upon the effective dose of each ingredient. Generally, an effective dose of each will be used. Thus, for example, when the compounds provided herein are used in combination with the second drug, or a composition containing such other drug, the weight ratio of the particulates to the second drug can range from about 1,000:1 to about 1:1,000, or about 200:1 to about 1:200.

Combinations of the particulates provided herein and other active ingredients will generally also be within the aforementioned range, but in each case, an effective dose of each active ingredient should be used.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

Cannabinoids and derivatives thereof are prepared in U.S. application Ser. No. 18/403,509, filed Jan. 3, 2024, and incorporated herein by reference.

### Example 1: A Randomized, Blinded, Placebo-Controlled Study to Evaluate the Efficacy and Tolerability of CBN at 25 mg, 50 mg, and 100 mg Daily Oral Doses, and Melatonin at 4 mg Single Daily Dose for Sleep Quality

The primary aim of this study was to assess the effects of three different formulations of TruCBN softgels and a formulation of 4 mg melatonin on sleep quality, relative to placebo control. There were 5 arms within the study; (1) 4 mg melatonin, (2) 25 mg CBN, (3) 50 mg CBN, (4) 100 mg CBN, and (5) placebo (see Table 1 for further details on study arm formulations). All study arms provided the test article as a softgel. Participants were instructed to take 1 softgel 1-2 hours before bed. To ensure that results were not biased, participants were randomly assigned to product, were blinded to the contents, and were not told what they were taking until the conclusion of the study. The secondary aim of the study was to assess the tolerability of the three formulations of TruCBN softgels and 4 mg melatonin, relative to placebo.

### Study Design

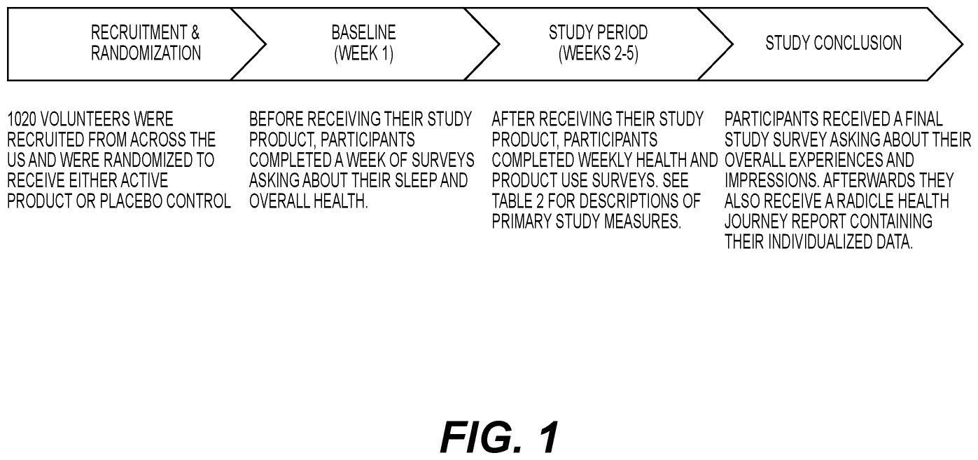

Study participants were at least 21 years old, endorsed a desire for better sleep quality, and expressed interest in taking a cannabinoid product to improve their sleep. The study was entirely virtual; participants were recruited through online recruitment channels, study product was delivered directly to participants' homes, and all data were collected via online surveys delivered to their mobile device or computer. There were four phases of the study (see FIG. 1 for study timeline): (1) Recruitment and Randomization, (2) Baseline (1 week), (3) Study period (2-5 weeks), and (4) Conclusion.

1a. Recruitment:

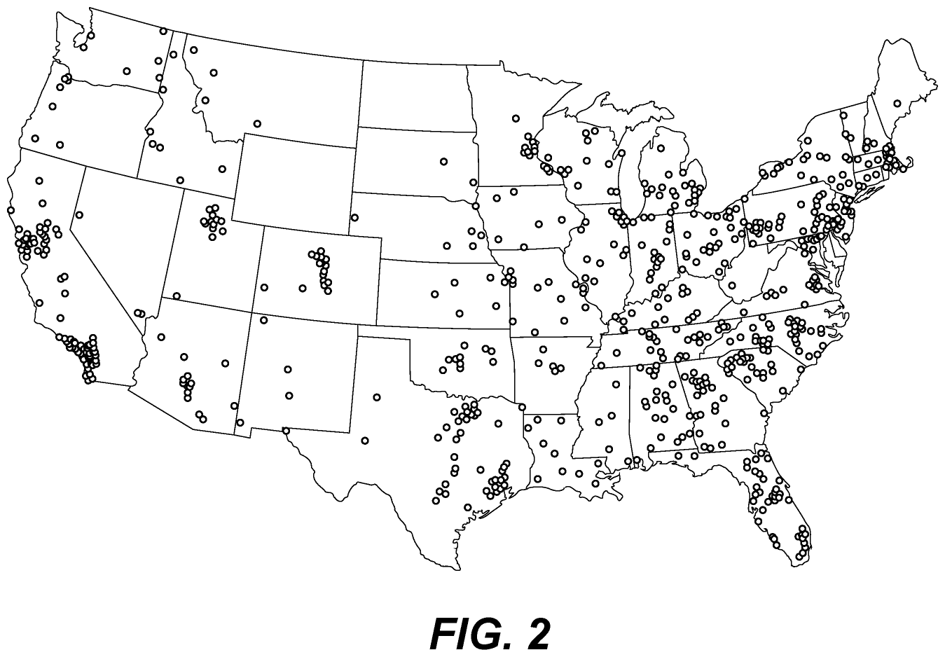

The total number of study participants was 1020, of which 54% were female, 46% were male, and 80% were white (see Table 1 for study demographics). The majority of participants (79%) reported that they suffered from sleep disturbances. Participant volunteers were recruited from across the United States (see FIG. 2 for participant map).

1b. Randomization:

Study participants were randomly divided into five study arms, each assigned to receive a different test article according to the Table 1.

TABLE 1
Study arms

| Study Arm | Number of participants |
|---|---|
| 4 mg melatonin | 202 |
| 25 mg CBN | 206 |
| 50 mg CBN | 205 |
| 100 mg CBN | 203 |
| placebo | 204 |

### 2. Baseline

Prior to receiving study test article, each participant was asked to complete study surveys in order to establish their baseline scores. Two clinical scales were used, each consisting of a questionnaire:

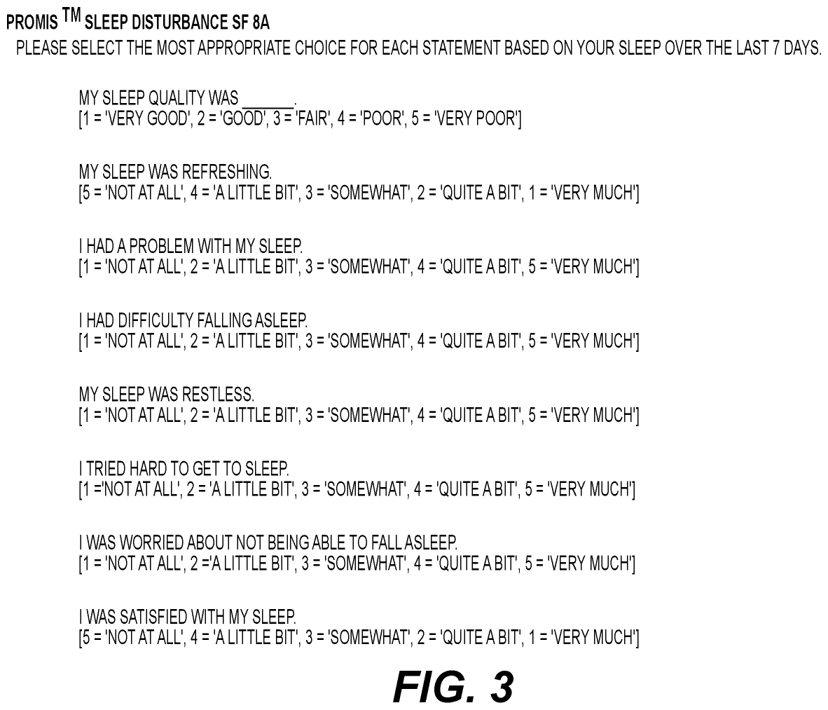

PROMIS™ Sleep Disturbance 8A: An 8-question survey, with severity thresholds based on T-scores not raw scores. The PROMIS™ Sleep Disturbance 8A survey questions are shown in FIG. 3. The severity scores are established as follows:
“None to slight”=Less than 55
“Mild”=55.0-59.9
“Moderate”=60.0-69.9
“Severe”=70 and up

The range of raw scores were from 8 to 40, with higher scores translating to greater sleep disturbance (i.e., worse sleep quality). Baseline values for the study participants are shown in Table 2. PROMIS™ Sleep Disturbance 8A threshold for minimally important difference: One-half the standard deviation of the baseline score (Purvis, Taylor E. et al., SPINE, 2018, 43(21):1512-1520).

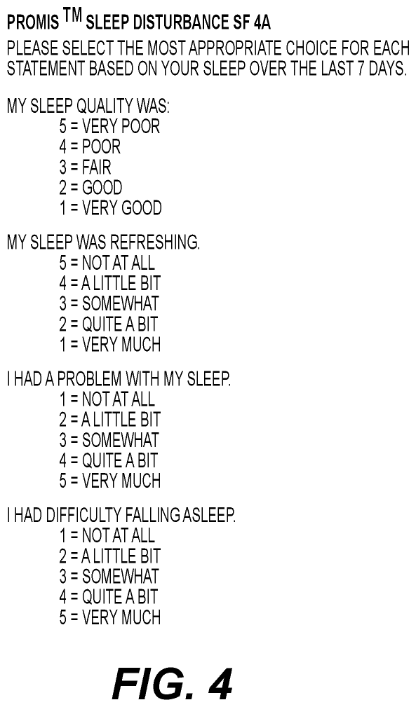

PROMIS™ Sleep Disturbance SF4A: a 4-question survey, with the questions shown in FIG. 4.

The PROMIS™ Sleep Disturbance SF4A threshold for minimally important difference: One-half the standard deviation of the baseline score (Norman, G. R. et al., Med. Care, 2003, 41:582-592).

TABLE 2
Participant characteristics and baseline scores

| | | | 4 mg | | 25 mg | | 50 mg | | 100 mg | | | |
|---|---|---|---|---|---|---|---|---|---|---|---|---|
| | Placebo | | Melatonin | | CBN | | CBN | | CBN | | Overall | |
| | N = 204 | | N = 202 | | N = 206 | | N = 205 | | N = 203 | | N = 1020 | |
| Age [mean (SD)] | 42.68 | (12.00) | 43.27 | (13.60) | 43.23 | (12.93) | 43.97 | (12.70) | 43.13 | (12.59) | 43.26 | (12.75) |
| BMI [mean (SD)] | 31.23 | (9.70) | 31.12 | (8.78) | 29.93 | (7.04) | 30.27 | (8.30) | 30.10 | (7.26) | 30.52 | (8.27) |

| | | | | | | |
|---|---|---|---|---|---|---|
| Unknown | 3 | 4 | 0 | 3 | 0 | 10 |

BMI category [N (%)]

| | | | | | | | | | | | | |
|---|---|---|---|---|---|---|---|---|---|---|---|---|
| Underweight | 1 | (0.5%) | 6 | (3.0%) | 1 | (0.5%) | 3 | (1.5%) | 1 | (0.5%) | 12 | (1.2%) |
| Normal weight | 47 | (23%) | 50 | (25%) | 50 | (24%) | 53 | (26%) | 47 | (23%) | 247 | (24%) |
| Overweight | 60 | (30%) | 42 | (21%) | 68 | (33%) | 65 | (32%) | 72 | (35%) | 307 | (30%) |
| Obesity | 93 | (46%) | 100 | (51%) | 87 | (42%) | 81 | (40%) | 83 | (41%) | 444 | (44%) |

| | | | | | | |
|---|---|---|---|---|---|---|
| Unknown | 3 | 4 | 0 | 3 | 0 | 10 |

Sex [N (%)]

| | | | | | | | | | | | | |
|---|---|---|---|---|---|---|---|---|---|---|---|---|
| Female | 111 | (54%) | 109 | (54%) | 112 | (54%) | 110 | (54%) | 110 | (54%) | 552 | (54%) |
| Male | 93 | (46%) | 93 | (46%) | 94 | (46%) | 95 | (46%) | 93 | (46%) | 468 | (46%) |

Race [N (%)]

| | | | | | | | | | | | | |
|---|---|---|---|---|---|---|---|---|---|---|---|---|
| White | 157 | (77%) | 171 | (85%) | 165 | (80%) | 165 | (80%) | 153 | (75%) | 811 | (80%) |
| Black | 20 | (9.8%) | 11 | (5.4%) | 14 | (6.8%) | 9 | (4.4%) | 15 | (7.4%) | 69 | (6.8%) |
| Multi-racial | 9 | (4.4%) | 11 | (5.4%) | 10 | (4.9%) | 10 | (4.9%) | 13 | (6.4%) | 53 | (5.2%) |
| Asian | 4 | (2.0%) | 2 | (1.0%) | 5 | (2.4%) | 12 | (5.9%) | 9 | (4.4%) | 32 | (3.1%) |
| Some other race | 7 | (3.4%) | 2 | (1.0%) | 9 | (4.4%) | 1 | (0.5%) | 6 | (3.0%) | 25 | (2.5%) |
| Prefer not to say | 2 | (1.0%) | 2 | (1.0%) | 3 | (1.5%) | 2 | (1.0%) | 3 | (1.5%) | 12 | (1.2%) |
| Unknown | 3 | (1.5%) | 3 | (1.5%) | 0 | (0%) | 3 | (1.5%) | 0 | (0%) | 9 | (0.9%) |
| American Indian or Alaska Native | 1 | (0.5%) | 0 | (0%) | 0 | (0%) | 2 | (1.0%) | 4 | (2.0%) | 7 | (0.7%) |
| Native Hawaiian or Pacific Islander | 1 | (0.5%) | 0 | (0%) | 0 | (0%) | 1 | (0.5%) | 0 | (0%) | 2 | (0.2%) |

Hispanic, LatinX, or Spanish origin [N (%)]

| | | | | | | | | | | | | |
|---|---|---|---|---|---|---|---|---|---|---|---|---|
| No | 183 | (90%) | 180 | (89%) | 185 | (90%) | 179 | (87%) | 183 | (90%) | 910 | (89%) |
| Yes | 18 | (8.8%) | 19 | (9.4%) | 18 | (8.7%) | 22 | (11%) | 17 | (8.4%) | 94 | (9.2%) |
| Prefer not to say | 3 | (1.5%) | 3 | (1.5%) | 3 | (1.5%) | 4 | (2.0%) | 3 | (1.5%) | 16 | (1.6%) |

Education level [N (%)]

| | | | | | | | | | | | | |
|---|---|---|---|---|---|---|---|---|---|---|---|---|
| Less than high school | 1 | (0.5%) | 4 | (2.0%) | 5 | (2.4%) | 0 | (0%) | 2 | (1.0%) | 12 | (1.2%) |
| High school diploma, no college | 30 | (15%) | 32 | (16%) | 33 | (16%) | 20 | (9.9%) | 21 | (10%) | 136 | (13%) |
| Some college, no degree | 43 | (21%) | 51 | (26%) | 56 | (27%) | 55 | (27%) | 57 | (28%) | 262 | (26%) |
| Trade/technical/vocational degree | 12 | (6.0%) | 15 | (7.5%) | 17 | (8.3%) | 15 | (7.4%) | 9 | (4.4%) | 68 | (6.7%) |
| Bachelors or associates degree | 80 | (40%) | 70 | (35%) | 69 | (33%) | 85 | (42%) | 83 | (41%) | 387 | (38%) |
| Masters or professional degree | 35 | (17%) | 27 | (14%) | 26 | (13%) | 26 | (13%) | 31 | (15%) | 145 | (14%) |
| Prefer not to say | 0 | (0%) | 0 | (0%) | 0 | (0%) | 1 | (0.5%) | 0 | (0%) | 1 | (<0.1%) |

| | | | | | | |
|---|---|---|---|---|---|---|
| Unknown | 3 | 3 | 0 | 3 | 0 | 9 |

PROMIS sleep baseline category [N (%)]

| | | | | | | | | | | | | |
|---|---|---|---|---|---|---|---|---|---|---|---|---|
| Within normal limits | 47 | (23%) | 48 | (24%) | 42 | (20%) | 42 | (20%) | 35 | (17%) | 214 | (21%) |
| Mild | 53 | (26%) | 51 | (25%) | 50 | (24%) | 47 | (23%) | 51 | (25%) | 252 | (25%) |
| Moderate | 82 | (40%) | 82 | (41%) | 88 | (43%) | 93 | (45%) | 82 | (40%) | 427 | (42%) |
| Severe | 22 | (11%) | 21 | (10%) | 26 | (13%) | 23 | (11%) | 35 | (17%) | 127 | (12%) |

PROMIS stress baseline category [N (%)]

| | | | | | | | | | | | | |
|---|---|---|---|---|---|---|---|---|---|---|---|---|
| Within normal limits | 28 | (14%) | 20 | (9.9%) | 26 | (13%) | 13 | (6.3%) | 15 | (7.4%) | 102 | (10%) |
| Mild | 32 | (16%) | 34 | (17%) | 34 | (17%) | 44 | (21%) | 35 | (17%) | 179 | (18%) |
| Moderate | 118 | (58%) | 110 | (54%) | 107 | (52%) | 114 | (56%) | 109 | (54%) | 558 | (55%) |
| Severe | 26 | (13%) | 38 | (19%) | 39 | (19%) | 34 | (17%) | 44 | (22%) | 181 | (18%) |

PROMIS anxiety baseline category [N (%)]

| | | | | | | | | | | | | |
|---|---|---|---|---|---|---|---|---|---|---|---|---|
| Within normal limits | 137 | (67%) | 130 | (64%) | 139 | (67%) | 133 | (65%) | 136 | (67%) | 675 | (66%) |

BMI = Body Mass Index;
PEG = Pain, Enjoyment of Life and General Activity Scale

### 3. Study Period

After receiving study product, each participant was instructed to take 1 softgel each day orally 1-2 hours before bed and complete weekly health and product use surveys. This phase of the study lasted about 4 weeks.

### 4. Study Conclusion

Participants received a final study survey asking about their overall experiences and impressions. Afterwards they also receive a Report containing their individualized data.

### Data Analysis

For each outcome under study, a regression model was run to test whether there were significant differences in effect between the active arms and placebo. If the mixed effects regression model showed significant improvement for a given outcome, then a generalized linear model was run to compare the likelihood of experiencing a minimal clinically important difference (MCID) in this outcome. For precision, adjustments were made for sex, age, race, ethnicity, and body mass index (BMI) in every model. A secondary analyses was also run (correcting for multiple comparisons) to evaluate the significance of health score changes within individual active arms, and whether there were significant differences in the effect between active arms. Analysis was conducted using Stata 17 and R. An intent-to-treat analysis was run, meaning that all participants who were assigned product (aside from the aforementioned exclusions) were included in the analysis according to their product assignment, regardless of product adherence or study attrition. Visuals of the predicted marginal means of health scores, rather than the raw mean scores, were prepared. Predicted marginal means were calculated from the statistical models and better represent the true differences between arms adjusting for any potential imbalances across arms. A finding that was “significant” means that the likelihood of falsely rejecting the null hypothesis that there were no differences between groups was very small (less than 5%; i.e., the p-value was less than 0.05). In other words, when certain findings were said to be “significant” they are not considered the result of random variation.

### Results

The participation rate remained reasonably uniform across each of the study arms from week 1 to week 4, with response rates ranging from 39% to 49% (see Table 3). The majority of participants (67%) completed at least one follow-up health survey following baseline.

TABLE 3
Weekly survey completion rates by study arm

| | % partic- | % partic- | % partic- | % partic- |
|---|---|---|---|---|
| | ipation | ipation | ipation | ipation |
| Study Arm | Week 1 | Week 2 | Week 3 | Week 4 |
| Placebo | 49 | 42 | 38 | 39 |
| 4 mg melatonin | 50 | 49 | 43 | 38 |
| 25 mg CBN | 54 | 47 | 45 | 41 |
| 50 mg CBN | 44 | 43 | 41 | 41 |
| 100 mg CBN | 55 | 55 | 45 | 46 |

Product use also remained relatively consistent among all study arms. As can be seen in Table 4, from week 1 to week 4, the number of softgels consumed per week ranged from 1-1.7 per day.

TABLE 4
Softgels consumed per day by study week

Average number of softgels/day

| Study Arm | Week 1 | Week 4 |
|---|---|---|
| placebo | 1.1 | 1.0 |
| 4 mg melatonin | 1.0 | 1.0 |
| 25 mg CBN | 1.2 | 1.2 |
| 50 mg CBN | 1.2 | 1.1 |
| 100 mg CBN | 1.2 | 1.7 |

### Sleep Quality:

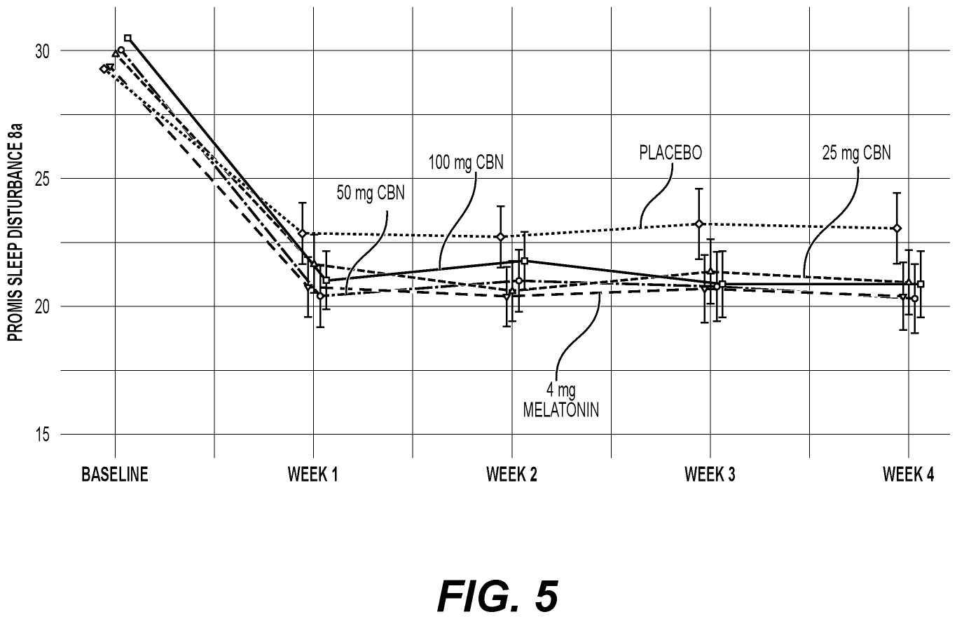

Tables 5-12 summarize the clinical findings using the PROMIS™ Sleep Disturbance 8A survey. There was a significant difference in the rate of mean PROMIS™ Sleep Disturbance 8A score change between 50 mg CBN and placebo and between 4 mg melatonin and placebo (see FIG. 5). There was a marginally significant difference in the rate of mean PROMIS™ Sleep Disturbance 8A score change between 25 mg CBN and placebo and between 100 mg CBN and placebo (see Table 5 for a summary of comparisons). As shown in Table 10, the estimated marginal percentage achieving meaningful clinical improvement on the PROMIS™ Sleep 8A was greatest in the 50 mg CBN arms of the study. Tables 13-17 summarize the clinical findings using the PROMIS™ Sleep Disturbance SF4A survey. As shown in Tables 15 and 16, the largest increase in Meaningful Clinical Improvement occurred in the 50 mg CBN arms of the study.

TABLE 5
Comparisons against placebo for PROMIS ™
Sleep Disturbance 8A score

| | Difference from | | t- | P |
|---|---|---|---|---|
| Study Arm | placebo (95% CL)¹ | | statistic² | value² |
| 4 mg melatonin − placebo | −2.67 | (−5.08, −0.25) | −2.71 | 0.025 |
| 25 mg CBN − placebo | −2.12 | (−4.47, 0.23) | −2.22 | 0.091 |
| 50 mg CBN − placebo | −2.74 | (−5.13, −0.34) | −2.81 | 0.019 |
| 100 mg CBN − placebo | −2.17 | (−4.5, 0.17) | −2.29 | 0.078 |

¹Contract calculated at week 4;
²Dunnett's test correction used to adjust for multiple comparisons

TABLE 6
Estimated marginal means¹ across time points
for PROMIS ™ Sleep Disturbance 8A

| | | 4 mg | 25 mg | 50 mg | 100 mg |
|---|---|---|---|---|---|
| Week | Placebo | melatonin | CBN | CBN | CBN |
| 1 | 22.8 | 20.7 | 21.6 | 20.4 | 20.99 |
| | (21.6, | (19.6, | (20.5, | (19.2, | (19.9, |
| | 24.0) | 21.9) | 22.8) | 21.6) | 22.1) |
| 2 | 22.7 | 20.4 | 20.6 | 21 | 21.77 |
| | (21.5, | (19.2, | (19.4, | (19.7, | (20.6, |
| | 23.9) | 21.6) | 21.7) | 22.2) | 22.9) |
| 3 | 23.2 | 20.7 | 21.3 | 20.7 | 20.84 |
| | (21.8, | (19.3, | (20.0, | (19.4, | (19.5, |
| | 24.6) | 22.0) | 22.6) | 22.1) | 22.1) |
| 4 | 23.0 | 20.4 | 20.9 | 20.3 | 20.9 |
| | (21.7, | (19, | (19.6, | (18.9, | (19.6, |
| | 24.4) | 21.7) | 22.2) | 21.6) | 22.1) |

¹Estimated marginal mean (95% confidence interval)

TABLE 7
Parameter estimates¹ for participant global impression
of change (PGIC) in sleep disturbance

| | | Standard | z | p |
|---|---|---|---|---|
| Parameter | Estimate | Error | Statistic | Value |
| Education Reference: | | | | |
| College degree | | | | |
| No College Degree | 0.28 | 0.16 | 1.71 | 0.087 |
| Sex Reference: Female | | | | |
| Male | −0.22 | 0.16 | −1.37 | 0.169 |
| Age | 0.00 | 0.01 | −0.68 | 0.498 |
| Race Reference: White | | | | |
| Non-White | 0.28 | 0.22 | 1.32 | 0.187 |
| BMI | −0.01 | 0.01 | −0.78 | 0.437 |
| Hispanic Reference: | | | | |
| Non-Hispanic | | | | |
| Hispanic | −0.08 | 0.30 | −0.29 | 0.775 |
| Treatment Reference: | | | | |
| Placebo | | | | |
| 4 mg Melatonin | 0.79 | 0.26 | 3.05 | 0.002 |
| 25 mg CBN | 0.63 | 0.26 | 2.42 | 0.015 |
| 50 mg CBN | 0.85 | 0.26 | 3.27 | 0.001 |
| 100 mg CBN | 0.85 | 0.25 | 3.35 | 0.001 |
| Intercepts | | | | |
| Much worse\|A little worse | −4.29 | 0.61 | −7.04 | 0.000 |
| A little worse\|No change | −2.48 | 0.48 | −5.13 | 0.000 |
| No change\|A little better | −0.25 | 0.46 | −0.54 | 0.590 |
| A little better\|Much better | 1.53 | 0.47 | 3.28 | 0.001 |

Parameters presented on log-odds scale

TABLE 8
Estimated odds ratio for sleep disturbance participants'
global impression of change (PGIC)

| | Odds Ratio (95% | | |
|---|---|---|---|
| Contrast | Confidence interval¹ | z-statistic | p Value¹ |
| 4 mg melatonin − placebo | 2.21 (1.15, 4.21) | 3.05 | 0.009 |
| 25 mg CBN − placebo | 1.88 (0.98, 3.62) | 2.42 | 0.054 |
| 50 mg CBN − placebo | 2.35 (1.22, 4.51) | 3.27 | 0.004 |
| 100 mg CBN − placebo | 2.35 (1.24, 4.44) | 3.35 | 0.003 |

¹Adjusted for multiple comparisons using Dunnett's test correction

TABLE 9
Estimated margins means for sleep disturbance participants'
global impression of change (PGIC)

| Study Arm | Marginal mean (95% confidence interval) | |
|---|---|---|
| placebo | 3.51 | (3.29, 3.72) |
| 4 mg melatonin | 3.88 | (3.67, 4.09) |
| 25 mg CBN | 3.81 | (3.59, 4.02) |
| 50 mg CBN | 3.91 | (3.7, 4.11) |
| 100 mg CBN | 3.91 | (3.7, 4.12 |

TABLE 10
Estimated marginal percentage achieving meaningful clinical
improvement on the PROMIS ™ Sleep 8A

| Study Arm | Estimated Marginal Mean¹ |
|---|---|
| placebo | 35.4% (25.22%, 49.55%) |
| 4 mg melatonin | 50.5% (37.54%, 67.94%) |
| 25 mg CBN | 47.1% (35.41%, 62.66%) |
| 50 mg CBN | 51.6% (38.93%, 68.48%) |
| 100 mg CBN | 49.1% (36.96%, 65.24%) |

¹Reflects estimated percentage of participants achieving meaningful clinical improvement with 95% confidence intervals in parentheses; generalized linear model assuming a Poisson distribution with a robust error variance; clinically meaningful improvement reflects a 30% or greater reduction from baseline

TABLE 11
Observed percentage achieving meaningful clinical
improvement on the PROMIS Sleep 8A

| Study Arm | % Achieving Meaningful Clinical Improvement¹ |
|---|---|
| placebo | 36.2 |
| 4 mg melatonin | 51.9 |
| 25 mg CBN | 46.7 |
| 50 mg CBN | 53.6 |
| 100 mg CBN | 50.0 |

¹Clinically meaningful improvement reflects a 30% or greater reduction from baseline

TABLE 12
Comparisons between study arms for meaningful clinical improvement
on the PROMIS ™ Sleep Disturbance 8A

| Study Arm | Relative Risk | z-test^{1, 2} | p-value² |
|---|---|---|---|
| 4 mg melatonin − placebo | 1.43 (0.7, 2.16) | 1.60 | 0.434 |
| 25 mg CBN − placebo | 1.33 (0.65, 2.01) | 1.33 | 0.729 |
| 50 mg CBN − placebo | 1.46 (0.72, 2.2) | 1.71 | 0.348 |
| 100 mg CBN − placebo | 1.39 (0.7, 2.08) | 1.54 | 0.498 |

¹Reflects ratio of probability of achieving a clinically meaningful improvement with 95% Confidence intervals in parentheses;
²Bonferroni correction used to adjust for multiple Comparisons; Generalized linear model assuming a Poisson distribution with a robust error variance; clinically meaningful improvement reflects a 30% or greater reduction from baseline

TABLE 13
Comparisons against placebo for PROMIS ™ Sleep
Disturbance SF4A score

| | Difference from | | | |
|---|---|---|---|---|
| Study Arm Contrast¹ | placebo (95% CL)² | | t-statistic² | P value² |
| 4 mg melatonin − placebo | −1.21 | (−2.39, −0.03) | −2.52 | 0.043 |
| 25 mg CBN − placebo | −0.9 | (−2.05, 0.24) | −1.94 | 0.169 |
| 50 mg CBN − placebo | 1.43 | (−2.6, −0.27) | −1.94 | 0.010 |
| 100 mg CBN − placebo | −0.98 | (−2.12, 0.16) | −2.12 | 0.115 |

¹Contrast calculated at week 4;
²Dunnett's test correction used to adjust for multiple comparison

TABLE 14
Estimated marginal means across time points for
PROMIS ™ Sleep Disturbance SF4A

| | | 4 mg | 25 mg | 50 mg | 100 mg |
|---|---|---|---|---|---|
| Week | Placebo¹ | melatonin | CBN | CBN | CBN |
| 1 | 11.8 | 10.8 | 11.0 | 10.7 | 10.8 |
| | (11.2, | (10.2, | (10.4, | (10.1, | (10.2, |
| | 12.4) | 11.4) | 11.5) | 11.3) | 11.4) |
| 2 | 11.8 | 10.7 | 10.81 | 10.9 | 11.33 |
| | (11.1, | (10.1, | (10.2, | (10.3, | (10.8, |
| | 12.4) | 11.3) | 11.4) | 11.5) | 11.9) |
| 3 | 11.8 | 10.7 | 11.1 | 10.8 | 10.8 |
| | (11.0, | (10.1, | (10.4, | (10.1, | (10.2, |
| | 12.5) | 11.4) | 11.7) | 11.5) | 11.5) |
| 4 | 11.8 | 10.5 | 10.9 | 10.3 | 10.8 |
| | (11.1, | (9.9, | (10.2, | (9.7, | (10.2, |
| | 12.4) | 11.2) | 11.5) | 11.0) | 11.4) |

¹Estimated marginal mean (95% confidence interval)

TABLE 15
Estimated marginal percentage achieving meaningful clinical
improvement on the PROMIS ™ Sleep SF4A

| Study Arm | Estimated Marginal Mean¹ |
|---|---|
| placebo | 34.38% (24.42%, 48.4%) |
| 4 mg melatonin | 51.25% (38.25%, 68.7%) |
| 25 mg CBN | 46.28% (34.73%, 61.7%) |
| 50 mg CBN | 54.76% (41.80%, 71.7%) |
| 100 mg CBN | 50.56% (38.37%, 66.6%) |

¹Reflects estimated percentage of participants achieving meaningful clinical improvement with 95% confidence intervals in parentheses; generalized linear model assuming a Poisson distribution with a robust error variance; clinically meaningful improvement reflects a 30% or greater reduction from baseline

TABLE 16
Observed percentage achieving meaningful clinical improvement
on the PROMIS ™ Sleep SF4A

| Study Arm | % Achieving Meaningful Clinical Improvement¹ |
|---|---|
| placebo | 36.25 |
| 4 mg melatonin | 53.25 |
| 25 mg CBN | 47.83 |
| 50 mg CBN | 58.33 |
| 100 mg CBN | 53.19 |

¹Clinically meaningful improvement reflects a 30% or greater reduction from baseline

TABLE 17
Comparisons between study arms for meaningful clinical
improvement on the PROMIS ™ Sleep SF4A

| Study Arm | Relative Risk | z-test^{1, 2} | p-value² |
|---|---|---|---|
| 4 mg melatonin − placebo | 1.49 (0.74, 2.25) | 1.78 | 0.302 |
| 25 mg CBN − placebo | 1.35 (0.66, 2.03) | 1.38 | 0.673 |
| 50 mg CBN − placebo | 1.59 (0.81, 2.37) | 2.08 | 0.149 |
| 100 mg CBN − placebo | 1.47 (0.75, 2.19) | 1.78 | 0.297 |

¹Reflects ratio of probability of achieving a clinically meaningful improvement with 95% Confidence intervals in parentheses;
²Bonferroni correction used to adjust for multiple Comparisons; Generalized linear model assuming a Poisson distribution with a robust error variance; clinically meaningful improvement reflects a 30% or greater reduction from baseline

Tables 18 and 19 summarize the clinical findings using the PROMIS™ Sleep Disturbance 8A survey.

Our analyses above reveal a significant difference in the rate of mean PROMIS Sleep Disturbance 8a score change between placebo and all of the active ingredient groups, including 25 mg CBN, 50 mg CBN, 100 mg CBN, and 4 mg Melatonin (FIG. 5 and Table 18).

TABLE 18
Parameter Estimates for PROMISTM Sleep Disturbances 8a

| | | Standard | | t | p |
|---|---|---|---|---|---|
| Parameter | Estimate | Error | df | Statistic | Value |
| (intercept) | 25.06 | 1.04 | 1849 | 24.06 | 0.000 |
| Education | | | | | |
| Reference: | | | | | |
| College | | | | | |
| degree | | | | | |
| No College | 1.50 | 0.36 | 1003 | 4.10 | 0.000 |
| Degree | | | | | |
| Prefer not to say | −4.38 | 5.35 | 1003 | −0.82 | 0.414 |
| Sex | | | | | |
| Reference: | | | | | |
| Female | | | | | |
| Male | −0.68 | 0.36 | 1003 | −1.88 | 0.061 |
| Age | −0.01 | 0.01 | 1003 | −0.50 | 0.619 |
| Race | | | | | |
| Reference: | | | | | |
| White | | | | | |
| Non-White | −0.61 | 0.48 | 1003 | −1.28 | 0.199 |
| Prefer not to say | 0.16 | 1.64 | 1003 | 0.10 | 0.923 |
| BMI | 0.08 | 0.02 | 103 | 3.68 | 0.000 |
| Hispanic | | | | | |
| Reference: | | | | | |
| Non-Hispanic | | | | | |
| Hispanic | −.79 | 0.64 | 1003 | −1.23 | 0.220 |
| Study Week | −1.08 | 0.18 | 1849 | −5.91 | 0.000 |
| Treatment | | | | | |
| Reference: | | | | | |
| Placebo | | | | | |
| 4 mg | −0.56 | 0.61 | 1003 | −0.92 | 0.358 |
| Melatonin | | | | | |
| 25 mg CBN | 0.14 | 0.60 | 1003 | 0.23 | 0.820 |
| 50 mg CBN | 0.28 | 0.61 | 1003 | 0.47 | 0.639 |
| 100 mg CBN | 0.59 | 0.60 | 1003 | 0.98 | 0.328 |
| Study Week * | | | | | |
| Treatment | | | | | |
| Reference: | | | | | |
| Placebo | | | | | |
| Study Week* | −0.58 | 0.26 | 1849 | −2.28 | 0.023 |
| 4 mg | | | | | |
| Melatonin | | | | | |
| Study Week* | −0.56 | 0.25 | 1849 | −2.26 | 0.024 |
| 25 mg CBN | | | | | |
| Study Week* | −0.67 | 0.25 | 1849 | −2.62 | 0.009 |
| 50 mg CBN | | | | | |
| Study Week* | −0.60 | 0.25 | 1849 | −2.41 | 0.016 |
| 100 mg CBN | | | | | |

Linear mixed-effects with random intercept at the participant level and a random-slope for study week.

The data collected above was analyzed post hoc to compare the difference between active arms containing CBN and melatonin (Table 2). Contrast analyses revealed no statistically significant differences in effects between 4 mg Melatonin and 25 mg CBN (t=0.32, p>0.05), 4 mg Melatonin and 50 mg CBN (t=−0.09, p>0.05), or 4 mg Melatonin and 100 mg CBN (t=0.06, p>0.05).

TABLE 19
Pairwise comparisons between study arms for
PROMIS ™ Sleep Disturbance 8A

| | Estimate (95% | | | |
|---|---|---|---|---|
| | confidence | | t- | P |
| Contrast | interval)¹ | | statistic | value¹ |
| 4 mg melatonin − 25 mg CBN | 0.08 | (−0.53, 0.69) | 0.32 | 1 |
| 4 mg melatonin − 50 mg CBN | −0.02 | (−0.62, 0.57) | −0.09 | 1 |
| 4 mg melatonin − 100 mg CBN | 0.01 | (−.058, 0.61) | 0.06 | 1 |

¹Contract calculated at week 4;
²Dunnett's test correction used to adjust for multiple comparisons

### Minimal Clinically Important Difference (MCID):

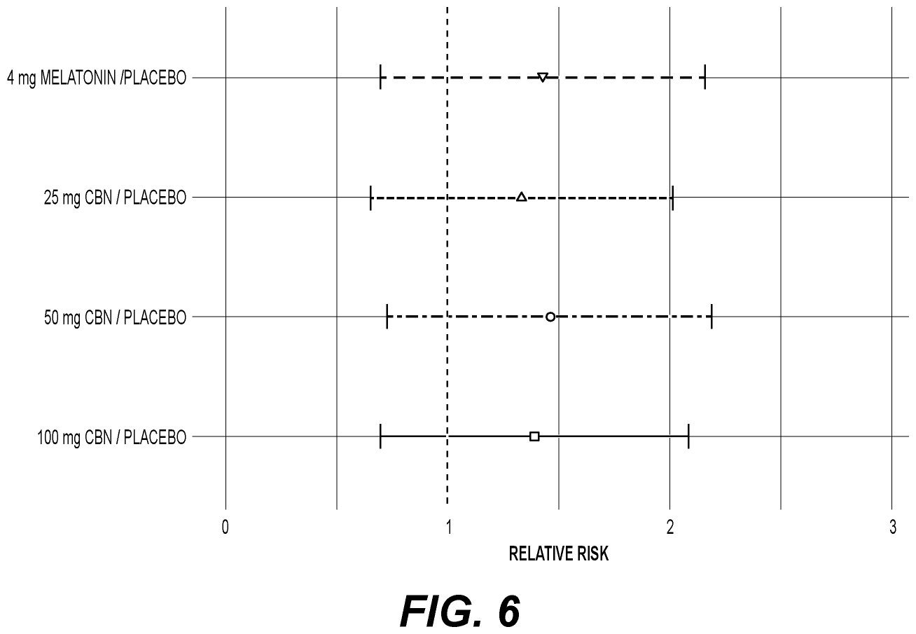

There were no significant differences in the likelihood of achieving a minimum clinically important difference (MCID) in the PROMIS™ Sleep Disturbance 8A score between any of the arms compared to placebo (see Table 12). Unlike FIG. 5, which demonstrated that average change in Sleep Disturbance score between two arms compared to placebo was significant, FIG. 6 focuses on the amount of change participants experienced. To reach MCID on the Sleep Disturbance scale, participants' scores had to change at least one-half the standard deviation of the baseline score. Therefore, while some arms experienced average change that was significantly higher than placebo, the amount of change did not reach the threshold for MCID. The relative risk ratio of 1 shown as the dotted line in FIG. 6 indicates the point at which there is no difference in risk/likelihood whereas a risk ratio below 1 suggests that the risk/likelihood of the product arm is lower than that of the reference group (placebo). It bears noting, that as shown in Table 18, about 36-53% of participants in every arm experienced a minimal clinically important improvement in their sleep disturbance, meaning that they experienced a meaningful change that could warrant a change in their symptom management.

TABLE 18
Proportion that experienced a minimal clinically important
difference (MCID)¹ in their PROMIS ™ Sleep
Disturbance 8A score, by study arm

| Study Arm | % experiencing MCID¹ |
|---|---|
| placebo | 36.2 |
| 4 mg melatonin | 51.9 |
| 25 mg CBN | 46.7 |
| 50 mg CBN | 53.6 |
| 100 mg CBN | 50.0 |

¹A score reduction of one half the standard deviation of the baseline score is considered an MCID

Participants reported experiencing an effect between 46 and 55 minutes after taking their study product. Table 19 shows the average time to effect.

TABLE 19
Average time to effect

| Study Arm | Time/min |
|---|---|
| placebo | 53.2 |
| 4 mg melatonin | 49.1 |
| 25 mg CBN | 54.9 |
| 50 mg CBN | 55.5 |
| 100 mg CBN | 46.4 |

### Tolerability:

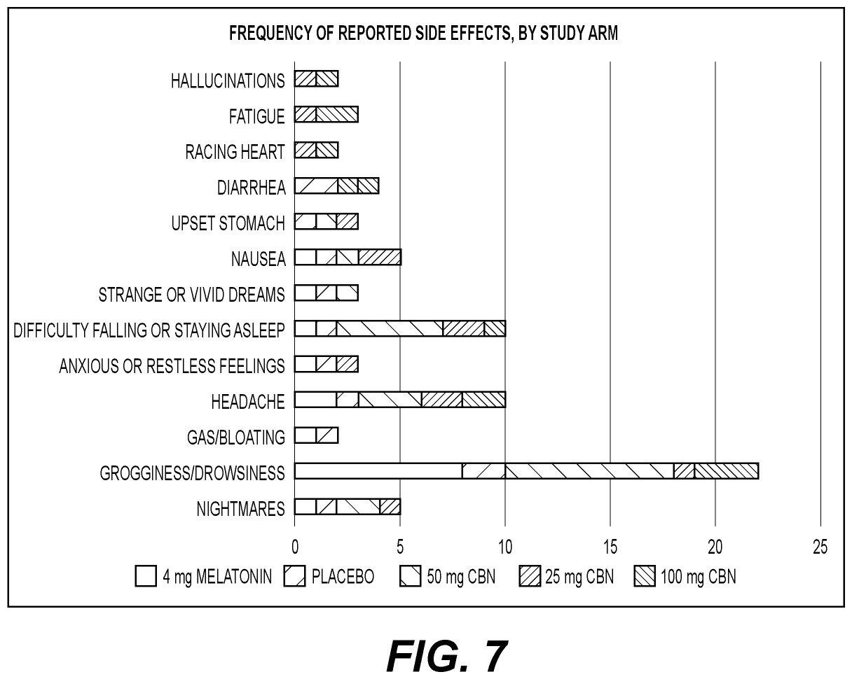

The side effects for all study arms were similar in frequency and mostly mild; none were considered serious or required use of emergency or non-emergency healthcare services. The percent incidence of side effects by study are listed in Table 20. The most commonly reported side effect was grogginess or drowsiness (see FIG. 7). Additional side effects reported by some participants included: chest pain, constipation, urinary tract infection, tingling in lips, dry mouth, weight gain, dry or itchy eyes, low libido, rash, and brain fog.

TABLE 20
Side effect data by study arm

| Study Arm | % of participants reporting any side effects |
|---|---|
| placebo | 6.4 |
| 4 mg melatonin | 11.9 |
| 25 mg CBN | 6.8 |
| 50 mg CBN | 12.9 |
| 100 mg CBN | 7.9 |

### Summary of Results:

The study results showed a significant difference in effect on sleep between 50 mg CBN and placebo and a marginally significant difference in effect on sleep between 25 mg CBN and placebo and between 100 mg CBN and placebo. The foregoing results suggest that the 50 mg CBN product can offer both significant and clinically meaningful improvement for those who want to experience higher sleep quality. In this study, the effect of a 50 mg CBN Softgel formulation on sleep extended beyond the placebo. Interestingly, study arms with a lower dose of CBN (25 mg) and higher dose of CBN (100 mg) also led to greater sleep quality improvement over placebo, though these differences in effect were not statistically significant. These results suggest that 50 mg CBN may be sufficient to induce significant therapeutic effects. All side effects were mild or moderate. There were no significant differences in the frequency of reported side effects between the active and placebo arms.

### Exemplary Embodiments

1. A method of reducing sleep disturbances in a subject, comprising administering a composition comprising about 40 mg to about 60 mg cannabinol to the subject, wherein the composition comprises about 0.5 wt % or less of cannabidiol and about 0.5 wt % or less of Δ9(10)-THC.
2. The method of embodiment 1, wherein the composition is substantially free of cannabidiol.
3. The method of embodiment 1 or 2, wherein the composition is substantially free of THC.
4. The method of any one of embodiments 1-3, wherein the composition is substantially free of Δ9(10)-THC.
5. The method of any one of embodiments 1-4, wherein the composition comprises about 0.5 wt % or less of additional cannabinoids.
6. The method of any one of embodiments 1-5, wherein the composition is substantially free of additional cannabinoids.
7. The method of embodiment 5 or 6, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H2CBND), dihydrocannabidiol (H2CBD), tetrahydrocannabidiol (H4CBD), Δ8(9)-iso-tetrahydrocannabinol (Δ8(9)-iso-THC), Δ4(8)-iso-tetrahydrocannabinol (Δ4(8)-iso-THC), Δ4(5)-iso-tetrahydrocannabinol (Δ4(5)-iso-THC), hexahydrocannabinol (HHC), Δ8(9)-tetrahydrocannabinol (Δ8(9)-THC), Δ9(10)-tetrahydrocannabinol (Δ9(10)-THC), Δ9(10)-tetrahydrocannabinolic acid (Δ9(10)-THCA), Δ9(10)-tetrahydrocannabinovarol (Δ9(10)-THCV), Δ10(10a)-tetrahydrocannabinol (Δ10(10a)-THC), Δ6a(10a)-tetrahydrocannabinol (Δ6a(10a)-THC), tetrahydrocannabigerol (H4CBG), and combinations thereof.
8. The method of any one of embodiments 1-7, wherein the cannabinol has a purity of at least about 95%, at least about 96%, at least about 97%, at least about 98%, at least about 99%, or at least about 99.9%.
9. The method of any one of embodiments 1-8, wherein the composition comprises about 50 mg of cannabinol.
10. The method of any one of embodiments 1-9, wherein the composition further comprises a pharmaceutical excipient.
11. The method of any one of embodiments 1-10, wherein the subject exhibits one or more risk factors or symptoms associated with lack of sleep or a sleep disorder.
12. The method of embodiment 11, wherein the sleep disorder is selected from the group of: insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition 13. The method of any one of embodiments 1-12, wherein the subject is a mammal.
14. The method of any one of embodiments 1-13, wherein the subject is a human.
15. A method for treating a sleep disorder in a subject, comprising administering a composition comprising about 40 mg to about 60 mg cannabinol to the subject, wherein the composition comprises about 0.5 wt % or less of cannabidiol and about 0.5 wt % or less of Δ9(10)-THC.
16. The method of embodiment 15, wherein the composition is substantially free of cannabidiol.
17. The method of embodiment 15 or 16, wherein the composition is substantially free of THC.
18. The method of any one of embodiments 15-17, wherein the composition is substantially free of Δ9(10)-THC.
19. The method of any one of embodiments 15-18, wherein the composition comprises about 0.5 wt % or less of additional cannabinoids.
20. The method of any one of embodiments 15-19, wherein the composition is substantially free of additional cannabinoids.
21. The method of embodiment 19 or 20, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H2CBND), dihydrocannabidiol (H2CBD), tetrahydrocannabidiol (H4CBD), Δ8(9)-iso-tetrahydrocannabinol (Δ8(9)-iso-THC), Δ4(8)-iso-tetrahydrocannabinol (Δ4(8)-iso-THC), Δ4(5)-iso-tetrahydrocannabinol (Δ4(5)-iso-THC), hexahydrocannabinol (HHC), Δ8(9)-tetrahydrocannabinol (Δ8(9)-THC), Δ9(10)-tetrahydrocannabinol (Δ9(10)-THC), Δ9(10)-tetrahydrocannabinolic acid (Δ9(10)-THCA), Δ9(10)-tetrahydrocannabinovarol (Δ9(10)-THCV), Δ10(10a)-tetrahydrocannabinol (Δ10(10a)-THC), Δ6a(10a)-tetrahydrocannabinol (Δ6a(10a)-THC), tetrahydrocannabigerol (H4CBG), and combinations thereof.
22. The method of any one of embodiments 15-21, wherein the cannabinol has a purity of at least about 95%, at least about 96%, at least about 97%, at least about 98%, at least about 99%, or at least about 99.9%.
23. The method of any one of embodiments 15-22, wherein the composition comprises about 50 mg of cannabinol.
24. The method of any one of embodiments 15-23, wherein the composition further comprises a pharmaceutical excipient.
25. The method of any one of embodiments 15-24, wherein the subject exhibits one or more risk factors or symptoms associated with lack of sleep or a sleep disorder.
26. The method of any one of embodiments 15-25, wherein the sleep disorder is selected from the group of: insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition.
27. The method of any one of embodiments 15-26, wherein the subject is a mammal.
28. The method of any one of embodiments 15-27, wherein the subject is a human.
29. The method of any one of embodiments 1-28, wherein the subject experiences less sleep disturbances according to a PROMIS™ Sleep Disturbance 8a Test.
30. A dosage form comprising cannabinol in an amount of about 40 mg to about 60 mg, wherein the cannabinol has a purity of at least about 95%.
31. The dosage form of embodiment 30 comprising about 50 mg of cannabinol.
32. The dosage form of embodiment 30 or 31, wherein the cannabinol has a purity of at least about 96%, at least about 97%, at least about 98%, at least about 99%, or at least about 99.9%.
33. The dosage form of any one of embodiments 30-32, wherein the dosage form further comprises a pharmaceutical excipient.
34. The dosage form of any one of embodiments 30-33, wherein the cannabinol is substantially free of cannabidiol.
35. The dosage form of any one of embodiments 30-34, wherein the cannabinol is substantially free of THC.
36. The dosage form of any one of embodiments 30-35, wherein the cannabinol is substantially free of Δ9(10)-THC.
37. The dosage form of any one of embodiments 30-36, wherein the cannabinol comprises about 0.5 wt % or less of additional cannabinoids.
38. The dosage form of any one of embodiments 30-37, wherein the cannabinol is substantially free of additional cannabinoids.
39. The dosage form of embodiment 37 or 38, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H2CBND), dihydrocannabidiol (H2CBD), tetrahydrocannabidiol (H4CBD), Δ8(9)-iso-tetrahydrocannabinol (Δ8(9)-iso-THC), Δ4(8)-iso-tetrahydrocannabinol (Δ4(8)-iso-THC), Δ4(5)-iso-tetrahydrocannabinol (Δ4(5)-iso-THC), hexahydrocannabinol (HHC), Δ8(9)-tetrahydrocannabinol (Δ8(9)-THC), Δ9(10)-tetrahydrocannabinol (Δ9(10)-THC), Δ9(10)-tetrahydrocannabinolic acid (Δ9(10)-THCA), Δ9(10)-tetrahydrocannabinovarol (Δ9(10)-THCV), Δ10(10a)-tetrahydrocannabinol (Δ10(10a)-THC), Δ6a(10a)-tetrahydrocannabinol (Δ6a(10a)-THC), tetrahydrocannabigerol (H4CBG), and combinations thereof.
40. A method of reducing sleep disturbances comprising administering to a subject in need thereof a composition comprising about 25 mg to about 100 mg cannabinol or a pharmaceutically acceptable salt thereof, wherein the composition comprises about 0.5 wt % or less of cannabidiol and about 0.5 wt % or less of Δ⁹⁽¹⁰⁾-THC.
41. The method of embodiment 40, wherein the composition comprises about 40 mg to about 60 mg of cannabinol, or a pharmaceutically acceptable salt thereof.
42. The method of any one of embodiment 40, wherein the composition comprises about 25 mg of cannabinol, or a pharmaceutically acceptable salt thereof.
43. The method of embodiment 40 or 41, wherein the composition comprises about 50 mg of cannabinol, or a pharmaceutically acceptable salt thereof.
44. The method of embodiment 40, wherein the composition comprises about 100 mg of cannabinol, or a pharmaceutically acceptable salt thereof.
45. The method of any one of embodiments 40-44, wherein the composition is substantially free of cannabidiol.
46. The method of any one of embodiments 40-45, wherein the composition is substantially free of THC.
47. The method of any one of embodiments 40-46, wherein the composition is substantially free of Δ⁹⁽¹⁰⁾-THC.
48. The method of any one of embodiments 40-47, wherein the composition comprises about 0.5 wt % or less of additional cannabinoids.
49. The method of any one of embodiments 40-48, wherein the composition is substantially free of additional cannabinoids.
50. The method of embodiment 48 or 49, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN-Ome), cannabicitran (CBT), dehydrocannabifuran (DHCBF), dihydrocannabinodiol (H2CBND), dihydrocannabidiol (H2CBD), tetrahydrocannabidiol (H4CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), tetrahydrocannabigerol (H4CBG), and combinations thereof.
51. The method of any one of embodiments 40-50, wherein the cannabinol, or a pharmaceutically acceptable salt thereof, has a purity of at least about 90%.
52. The method of any one of embodiments 40-51, wherein the cannabinol, or a pharmaceutically acceptable salt thereof, has a purity of at least about 95%, at least about 96%, at least about 97%, at least about 98%, at least about 99%, or at least about 99.9%.
53. The method of any one of embodiments 40-52, wherein the composition further comprises a pharmaceutical excipient.
54. The method of any one of embodiments 40-53, wherein the subject exhibits one or more risk factors or symptoms associated with lack of sleep or a sleep disorder.
55. The method of embodiment 54, wherein the sleep disorder is selected from the group of: insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition.
56. The method of any one of embodiments 40-55, wherein the subject experiences less sleep disturbances according to a PROMIS™ Sleep Disturbance 8a Test after administering the cannabinol composition as compared to before administering the cannabinol composition.
57. The method of any one of embodiments 40-56, wherein the subject is a mammal.
58. The method of any one of embodiments 40-57, wherein the subject is a human.
59. A method for treating a sleep disorder in a subject, comprising administering a composition comprising about 25 mg to about 100 mg cannabinol, or a pharmaceutically acceptable salt thereof, to the subject, wherein the composition comprises about 0.5 wt % or less of cannabidiol and about 0.5 wt % or less of Δ⁹⁽¹⁰⁾-THC.
60. A method for treating a sleep disorder, comprising administering to a subject in need thereof a composition comprising about 25 mg to about 100 mg cannabinol or a pharmaceutically acceptable salt thereof, wherein the composition comprises about 0.5 wt % or less of cannabidiol and about 0.5 wt % or less of Δ⁹⁽¹⁰⁾-THC.
61. The method of embodiment 60, wherein the composition comprises about 40 mg to about 60 mg cannabinol or a pharmaceutically acceptable salt thereof.
62. The method of embodiment 60, wherein the composition comprises about 25 mg of cannabinol, or a pharmaceutically acceptable salt thereof.
63. The method of embodiment 60 or 61, wherein the composition comprises about 50 mg of cannabinol, or a pharmaceutically acceptable salt thereof.
64. The method of embodiment 60, wherein the composition comprises about 100 mg of cannabinol, or a pharmaceutically acceptable salt thereof.
65. The method of any one of embodiments 60-64, wherein the composition is substantially free of cannabidiol.
66. The method of any one of embodiments 60-65, wherein the composition is substantially free of THC.
67. The method of any one of embodiments 60-66, wherein the composition is substantially free of Δ⁹⁽¹⁰⁾-THC.
68. The method of any one of embodiments 60-67, wherein the composition comprises about 0.5 wt % or less of additional cannabinoids.
69. The method of any one of embodiments 60-68, wherein the composition is substantially free of additional cannabinoids.
70. The method of embodiment 68 or 69, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN-Oac), cannabinodiol (CBND), methoxycannabinol (CBN-Ome), cannabicitran (CBT), dehydrocannabifuran (DHCBF), dihydrocannabinodiol (H2CBND), dihydrocannabidiol (H2CBD), tetrahydrocannabidiol (H4CBD), Δ⁸⁽⁹⁾-iso-tetrahydrocannabinol (Δ⁸⁽⁹⁾-iso-THC), Δ⁴(8-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), tetrahydrocannabigerol (H4CBG), and combinations thereof.
71. The method of any one of embodiments 60-70, wherein the cannabinol, or a pharmaceutically acceptable salt thereof, has a purity of at least about 90%.
72. The method of any one of embodiments 60-71, wherein the cannabinol, or a pharmaceutically acceptable salt thereof, has a purity of at least about 95%, at least about 96%, at least about 97%, at least about 98%, at least about 99%, or at least about 99.9%.
73. The method of any one of embodiments 60-72, wherein the composition further comprises a pharmaceutical excipient.
74. The method of any one of embodiments 60-73, wherein the subject exhibits one or more risk factors or symptoms associated with lack of sleep or a sleep disorder.
75. The method of any one of embodiments 60-74, wherein the sleep disorder is selected from the group of: insomnia, narcolepsy, hypersomnia, sleep apnea, periodic limb movement disorder, restless legs syndrome, nocturnal eating (drinking) syndrome, jet lag, shift work sleep disorder, irregular sleep-wake pattern, confusional arousals, sleepwalking, sleep terrors, sleep talking, nightmares, sleep paralysis, REM sleep behavior disorder, snoring, sleeping sickness, obstructive sleep apnea (OSA), circadian rhythm disorders, or a sleep disorder associated with another disease or condition.
76. The method of any one of embodiments 60-75, wherein the subject experiences less sleep disturbances according to a PROMIS™ Sleep Disturbance 8a Test after administering the cannabinol composition as compared to before administering the cannabinol composition.
77. The method of any one of embodiments 60-76, wherein the subject is a mammal.
78. The method of any one of embodiments 60-77, wherein the subject is a human.
79. A dosage form comprising cannabinol, or a pharmaceutically acceptable salt thereof, in an amount of about 25 mg to about 100 mg, wherein the cannabinol, or pharmaceutically acceptable salt thereof, has a purity of at least about 90%. 80. The dosage form of embodiment 79 comprising about 40 mg to about 60 mg of cannabinol, or a pharmaceutically acceptable salt thereof.
81. The dosage form of embodiment 79 comprising about 25 mg of cannabinol, or a pharmaceutically acceptable salt thereof.
82. The dosage form of embodiment 79 or 80 comprising about 50 mg of cannabinol, or a pharmaceutically acceptable salt thereof.
83. The dosage form of embodiment 79 comprising about 100 mg of cannabinol, or a pharmaceutically acceptable salt thereof.
84. The dosage form of any one of embodiments 79-83, wherein the cannabinol, or a pharmaceutically acceptable salt thereof, has a purity of at least about 95%, at least about 96%, at least about 97%, at least about 98%, at least about 99%, or at least about 99.9%.
85. The dosage form of any one of embodiments 79-84, wherein the dosage form further comprises a pharmaceutical excipient.
86. The dosage form of any one of embodiments 79-85, wherein the cannabinol, or a pharmaceutically acceptable salt thereof, is substantially free of cannabidiol.
87. The dosage form of any one of embodiments 79-86, wherein the cannabinol, or a pharmaceutically acceptable salt thereof, is substantially free of THC.
88. The dosage form of any one of embodiments 79-87, wherein the cannabinol, or a pharmaceutically acceptable salt thereof, is substantially free of Δ⁹⁽¹⁰⁾-THC.
89. The dosage form of any one of embodiments 79-88, wherein the cannabinol, or a pharmaceutically acceptable salt thereof, comprises about 0.5 wt % or less of additional cannabinoids.
90. The dosage form of any one of embodiments 79-89, wherein the cannabinol, or a pharmaceutically acceptable salt thereof, is substantially free of additional cannabinoids.
91. The dosage form of embodiment 89 or 90, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN-Oac), cannabinodiol (CBND), methoxycannabinol (CBN-Ome), cannabicitran (CBT), dehydrocannabifuran (DHCBF), dihydrocannabinodiol (H2CBND), dihydrocannabidiol (H2CBD), tetrahydrocannabidiol (H4CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), and combinations thereof.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results.

1-25. (canceled)

25. A dosage form comprising cannabinol or a pharmaceutically acceptable salt thereof in an amount of about 25 mg to about 100 mg, wherein the cannabinol or pharmaceutically acceptable salt thereof, has a purity of at least about 90%.

26. The dosage form of claim 25, wherein the cannabinol or a pharmaceutically acceptable salt thereof has a purity of at least about 95%.

27. The dosage form of claim 25, wherein the dosage form comprises about 40 mg to about 60 mg of cannabinol or a pharmaceutically acceptable salt thereof.

28. The dosage form of claim 25, wherein the dosage form further comprises a pharmaceutical excipient.

29. The dosage form of claim 25, wherein the cannabinol or a pharmaceutically acceptable salt thereof is substantially free of cannabidiol.

30. The dosage form of claim 25, wherein the cannabinol or a pharmaceutically acceptable salt thereof is substantially free of THC.

31. The dosage form of claim 25 comprising about 25 mg of cannabinol, or a pharmaceutically acceptable salt thereof.

32. The dosage form of claim 25 comprising about 50 mg of cannabinol, or a pharmaceutically acceptable salt thereof.

33. The dosage form of claim 25 comprising about 100 mg of cannabinol, or a pharmaceutically acceptable salt thereof.

34. The dosage form of claim 25, wherein the cannabinol, or a pharmaceutically acceptable salt thereof, has a purity of at least about 98%.

35. The dosage form of claim 25, wherein the cannabinol, or a pharmaceutically acceptable salt thereof, is substantially free of Δ⁹⁽¹⁰⁾-THC.

36. The dosage form of claim 25, wherein the cannabinol, or a pharmaceutically acceptable salt thereof, comprises about 0.5 wt % or less of additional cannabinoids.

37. The dosage form of claim 25, wherein the cannabinol, or a pharmaceutically acceptable salt thereof, is substantially free of additional cannabinoids.

38. The dosage form of claim 37, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN-Oac), cannabinodiol (CBND), methoxycannabinol (CBN-Ome), cannabicitran (CBT), dehydrocannabifuran (DHCBF), dihydrocannabinodiol (H2CBND), dihydrocannabidiol (H2CBD), tetrahydrocannabidiol (H4CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), and combinations thereof.

39. A dosage form comprising cannabinol in an amount of about 40 mg to about 60 mg, wherein the cannabinol has a purity of at least about 95%.

40. The dosage form of claim 39 comprising about 50 mg of cannabinol.

41. The dosage form of claim 39, wherein the cannabinol has a purity of at least about 98%.

42. The dosage form of claim 39, wherein the dosage form further comprises a pharmaceutical excipient.

43. The dosage form of claim 39, wherein the cannabinol is substantially free of cannabidiol.

44. The dosage form of claim 39, wherein the cannabinol is substantially free of THC.

45. The dosage form of claim 39, wherein the cannabinol is substantially free of Δ⁹⁽¹⁰⁾-THC.

46. The dosage form of claim 39, wherein the cannabinol comprises about 0.5 wt % or less of additional cannabinoids.

47. The dosage form of claim 39, wherein the cannabinol is substantially free of additional cannabinoids.

48. The dosage form of claim 39, wherein the additional cannabinoids are selected from the group consisting of: cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H2CBND), dihydrocannabidiol (H2CBD), tetrahydrocannabidiol (H4CBD), Δ8(9)-iso-tetrahydrocannabinol (Δ8(9)-iso-THC), Δ4(8)-iso-tetrahydrocannabinol (Δ4(8)-iso-THC), Δ4(5)-iso-tetrahydrocannabinol (Δ4(5)-iso-THC), hexahydrocannabinol (HHC), Δ8(9)-tetrahydrocannabinol (Δ8(9)-THC), Δ9(10)-tetrahydrocannabinol (Δ9(10)-THC), Δ9(10)-tetrahydrocannabinolic acid (Δ9(10)-THCA), Δ9(10)-tetrahydrocannabinovarol (Δ9(10)-THCV), Δ10(10a)-tetrahydrocannabinol (Δ10(10a)-THC), Δ6a(10a)-tetrahydrocannabinol (Δ6a(10a)-THC), tetrahydrocannabigerol (H4CBG), and combinations thereof.

49. The dosage form of claim 34 comprising about 50 mg of cannabinol and wherein the dosage form is in the form of a soft gel capsule.

50. The dosage form of claim 41 comprising about 50 mg of cannabinol and wherein the dosage form is in the form of a soft gel capsule.

51. The dosage form of claim 25 comprising about 50 mg of cannabinol having a purity of at least 99% and wherein the dosage form is in the form of a soft gel capsule.

52. The dosage form of claim 39 comprising about 50 mg of cannabinol having a purity of at least 99% and wherein the dosage form is in the form of a soft gel capsule.
